(19) United States
(12) Patent Application Publication
(10) Pub. No.: US 2017/0191099 A1
(43) Pub. Date: Jul. 6, 2017

(54) SYSTEM AND METHOD FOR TREATMENT OF BIOMASS FOR THE PRODUCTION OF BIOFUELS AND BIOCHEMICALS

(71) Applicant: Arbiom Inc., Durham, NC (US)

(72) Inventors: William F. McDonald, Utica, OH (US); Naga Vinod Kumar Bathula, Norton, VA (US); Michael Bowyer, Bedford, VA (US); Brandon Kiser, Wise, VA (US); Vaishnavi Srinivasan, Norton, VA (US); Lisette Tenlep, Big Stone Gap, VA (US); Brie-Anna Walker, Wise, VA (US)

(73) Assignee: Arbiom Inc., Durham, NC (US)

(21) Appl. No.: 15/368,652

(22) Filed: Dec. 4, 2016

Related U.S. Application Data
(60) Provisional application No. 62/263,597, filed on Dec. 4, 2015.
(60) Provisional application No. 62/362,561, filed on Jul. 14, 2016.

(51) Int. Cl. C12P 19/02

(52) U.S. Cl. CPC C12P 19/02; C12P 2201/00

(57) ABSTRACT

A system and method to treat biomass to make bioproducts such as biofuels and biochemical is disclosed. The systems are configured to treat a liquid component separated from biomass to yield sugars available to form a fermentation product or bio-product. The system and method comprises treatment and separation of process streams (e.g. liquids, solids, etc.) to produce bioproducts such as fuel and biochemical as well as to recover/reuse and utilize content of process streams. Lignin is separated from the bioproduct stream (e.g. a solids component) forming a recovered bioproducts; efficient removal of lignin facilitates productive operation of the system. The system disclosed is configured to recover and recycle acid (e.g. phosphoric acid) used as a concentrated acid in the process to pre-treat biomass (for bioprocessing). The acid used in the process is recycled and recovered in order to establish economically attractive biorefinery to make bio-products from biomass (e.g. to reduce the cost of operation such as from supply of fresh acid). Concentrated acid used to pre-treat biomass may comprise about 90 to over 98 percent recovered acid (and 10 to less than 2 percent fresh acid). The recovery and recycle process for the acid (e.g. acid used to pre-treat biomass) may comprise one or more of the inventive concepts and features shown and described in the FIGURES and specification.

### RELATED APPLICATION

The present application is related to U.S. Provisional Patent Application No. 62/263,597 titled “System and Method for Treatment of Biomass for the Production of Biofuels and Biochemicals” filed Dec. 4, 2015.

### FIELD

The present invention relates to a system and method for treatment of biomass for the production of biofuels and biochemicals. The present invention also relates to a system and method for the pretreatment of biomass in the form of agricultural residues (e.g. corn stover, rape seed straw, wheat straw, oat straw, sorghum, sugar cane bagasse, etc.) woody substrates (e.g. saw dust, wood chips including both hard- and softwoods, forest thinning, etc.) and woody residues (e.g. from saw mills, furniture making, and home building). The present invention further relates to a system and method for pretreatment of biomass with acid via catalytic processes (e.g. chemical catalysis or biological conversion).

### BACKGROUND

It is well-known to use biomass as a feedstock for the production of biofuels and biochemicals.

Grain and other starch-based feedstocks can be used to produce biofuels and biochemicals. Such starch-based feedstocks include most grains (corn, milo, sorghum, wheat, oats, barley, millet, etc.), potatoes, cassava, and similar crops. Biomass (e.g. specifically lignocellulosic material that can be converted to sugars) can also be used to provide biofuels and biochemicals. Such biomass feedstocks include corn cobs, corn stover, wheat and oat straw, soy straw, rape seed straw, empty fruit bunches, hard wood, soft wood, palm wood, and any lignocellulosic substrate of sufficient quantity for conversion to a bio product.

Biomass comprises plant matter that can be used for direct conversion to fuel or energy or as a feedstock for conversion into another bio-product (e.g. biofuel or biochemical) produced at a bio-refinery similar to an ethanol plant. Biomass may comprise, for example, saw dust from a wood processor (lumber mill, hard wood flooring manufacturer, etc.) made available as the wood is mechanically processed into a product (e.g. including lumber, flooring, etc.). In order to be processed as a feedstock, the biomass must be delivered to the bio-refinery. The bio-refinery may be co-located adjacent the processor or at a separate site. For example, to use as a feedstock saw dust from a wood processor, the saw dust would be conveyed after collection and from storage to the bio-refinery. If the plants were not co-located, the biomass would be shipped via truck, train or similar shipping process to the bio-refinery location.

In a typical biofuel plant that uses a grain, the biofuel is produced from the conversion of starch. For example, the grain is milled to prepare the starch containing material for processing. By a well-known process, the material containing starch is slurried with water and liquefied either with dilute acid or an enzyme (amylase and glucoamylase) to saccharify the starch to sugar (e.g. glucose and fructose); then an organism (e.g. a yeast that converts the sugar to a fermentation product) is introduced into the slurry. Fermentation produces in the grain slurry a product that is commonly referred to as a “beer” that comprises a liquid component (containing the fermentation product) water, soluble components from the grain, minor fermentation products (e.g. acetic acid and glycerol), and a solids component containing unfermented or unreacted solid particles. The fermentation product is sent to a product recovery operation that might entail distillation, crystallization/recrystallization, and/or filtration. Once the fermentation product is recovered the residue that includes water and residual grain components is dried to recover the distillers grains for animal feed. There might be additional products that can be realized from the residue that could include oil, fiber, and protein. Waste streams such as water can be treated for reuse with in the plant.

In a bio-refinery designed to produce a biofuel or bio-product from biomass, the fermentative product is now produced (e.g. by chemical catalysis or biological conversion) from the hemicellulose and cellulose contained within the lignocellulosic material of the biomass. The lignocellulosic material contains or comprises cellulose hemicellulose, lignin, minerals, ash, and minor components including acetic acid. The glucan polysaccharide, cellulose, is comprised of hexose (C6) sugars, primarily glucose linked in linear chains. Hemicellulose is branched chain polysaccharide that is comprised of pentose (C5) sugar monomers including xylose and arabinose and smaller amounts of hexose sugar monomers including galactose and mannose. (The hemicellulose also contains acetate groups as branches off of the main polymer backbone.)

The lignocellulosic biomass is treated or prepared in a method intended to render the polysaccharide chains of the material susceptible to breakdown into component sugars (e.g. C5 and C6 sugars); the component sugars made available by treatment may be subsequently fermented or reacted to form a resulting fermentation product that can be recovered (e.g. a biofuel or bio-product). After fermentation or conversion, the fermentation product is sent to an operation in which the crude product is recovered then the crude material is able to be processed into a marketable final product (e.g. biofuel/bio-product). Other fractions of the biomass, for instance, lignin and organic acids may also be recovered as products or coproducts during the processing of the biomass to sugar and the sugar to a marketable product. The determination of how to efficiently prepare and treat the lignocellulosic material for production of a marketable product depends on the chemical composition and the type of biomass (e.g. agricultural residue or woody material). Lignocellulosic material from different crops or different woody materials will contain different amounts of hemicellulose, cellulose, lignin, organic acids, and ash. For example, the biological composition of corn stover is quite different from the biological composition of oak hard wood. Corn stover is significantly less difficult to process as a feedstock than oak hard wood.

Known processes for treating and using biomass such as woody material as a feedstock for bio-refinery are known. See, for example, International Patent Application Number PCT/US2006/011411 (publication number WO 2007/111605) filed Mar. 29, 2006, titled “Cellulose Solvent Based Lignocellulosic Fractionation with Modest Conditions and Reagent Cycling”; and International Patent Application Number PCT/US 2009/037234 (publication number WO 2009/114843) filed Mar. 16, 2009, titled “Method and Apparatus for Lignocellulose Pretreatment using a Super Cellulose Solvent and Highly Volatile Solvents”.

It would be advantageous to provide an improved system and method to pretreat biomass comprising lignocellulosic material to produce sugars that are then used to produce a fermentation product. A system to pretreat or treat biomass such as saw dust would be advantageous for the production of a marketable fermentation product. It would be further advantageous to provide a system for the pretreatment or treatment of saw dust that improves or facilitates the efficiency and yield of the cellulosic sugars to fermentation product. The advantageous improvement in efficiency includes the recovery of the acid and solvent from the pretreated biomass and recycling them back into the process in a way that improves the overall process economics to make the overall process more economically feasible.

### SUMMARY

The present invention relates to a system and method for treatment of biomass for the production of biofuels and biochemicals. The present invention also relates to a system and method for the pretreatment of biomass in the form of agricultural residues (corn stover, rape seed straw, wheat straw, oat straw, sorghum, sugar cane bagasse, etc.) and woody substrates (saw dust, wood chips including both hard- and softwoods, forest thinning, and woody residues from saw mills, furniture making, and home building). The present invention further relates to a system and method for pretreatment of biomass with acid for the production of biofuels and biochemicals via catalytic processes. These catalytic processes can be either chemical catalysis or biological conversion.

The present invention relates to a method to pretreat biomass to be used in a bio-refinery to produce a biofuel or biochemical using a fermentative process. The method comprises preparing the biomass into prepared biomass; and pre-treating the prepared biomass into a pretreated biomass by application of a concentrated acid having a concentration of about 75 percent to about 100 percent at a temperature of about 20 to about 90 degrees Celsius for a period of time in a range of about 15 minutes to 4 hours; wherein the fermentation product derived from the fermentation process can be obtained by separating the pretreated biomass into a liquid component comprising lignin, solvent, and acid and a solids component from which glucose can be made available for fermentation into a fermentation product; wherein the biomass comprises lignocellulosic material; and wherein the lignocellulosic material comprises hard wood sawdust consisting of at least oak wood and can also contain yellow poplar, maple, hickory, and other indigenous hard woods.

The present invention also relates to a system and method to pretreat biomass to produce sugars that are then used to produce a fermentation product. The method comprises the steps of preparing the biomass into a prepared biomass and pretreating the prepared biomass into a pretreated biomass by application of a concentrated acid, particularly phosphoric acid, having a concentration not less than 80 percent by weight at a temperature of 20 to 30 degrees Celsius for a period of 0 to 60 minutes providing a presoak for chemical impregnation into the biomass. The presoak or chemical impregnation step is followed by increasing the temperature to 30 to 80 degrees Celsius and maintaining that temperature for 10 to 120 minutes providing a “cook” time for the biomass. Once the cook time is completed the reaction is quenched and the cellulose and residual hemicellulose are precipitated by the addition of a suitable solvent, for instance ethanol or water. The cooked and quenched biomass is washed co-currently or counter-currently to extract the acid from the precipitated biomass. A suitable liquid solid separation process must be employed to enable this separation and extraction to occur, for instance, a plate and frame filter, a basket centrifuge, decanter centrifuge, screw press, settling and decanting, filter belts (including pressure and vacuum), etc. Once the acid is extracted, the cellulosic solids are transferred to the enzyme hydrolysis operation where they are exposed to an enzymatic process to break down the cellulose to the component sugars, specifically, glucose and xylose. The enzyme is available commercially through Novozymes (Franklinton N.C.). The biomass is comprised of hard wood saw dust dried to approximately 10 percent moisture.

The present invention relates to the pretreatment and the subsequent recovery and recycle of the acid once extracted from the pretreated biomass. Once the material containing the biomass, phosphoric acid, and water has been separated, the liquid phase is called the mother liquor or black liquor. The cellulosic solids are then washed repeatedly with ethanol and water or just water to finally extract the acid from the biomass creating a second liquid phase termed the wash liquors. This method of acid recovery further entails the use of an evaporation system that removes solvent (water or ethanol) from the acid (e.g. mother liquor or black liquor). The evaporation brings the acid content up from 10 to 30 percent by weight to an acid content greater than 80 percent. Once the acid is re-concentrated and cooled, residual solids can be precipitated from the acid by adding water or by adding water with a flocculating aid added. With ethanol as the quench solvent, the lignin remains soluble in the ethanol acid mixture causing this lignin to move forward with the acidic liquid phase. The ethanol is evaporated, the lignin precipitated by adding water and a flocculant and removed using a suitable filtration methodology (plate and frame filter, basket centrifuge, decanter centrifuge, etc.) creating a “clean” diluted acid stream. The diluted acid is re-concentrated using evaporation to achieve at least an 80 percent phosphoric acid solution by weight.

The present invention relates to a system and method to pretreat biomass to be used in a biorefinery to produce a biofuel or biochemical using a fermentation process. The system and method comprise (a) preparing the biomass into prepared biomass; (b) pre-treating the prepared biomass into a pretreated biomass by application of a concentrated acid; (c) facilitating the production of a fermentation product derived from the biomass to be obtained by separating the pretreated biomass into (1) a liquid component comprising lignin, solvent and acid and (2) a solids component from which glucose can be made available for fermentation into the fermentation product; (d) recovering recoverable acid from the pre-treated biomass; (e) producing recovered acid from recoverable acid; and (f) contributing recovered acid to be used as concentrated acid. The biomass comprises lignocellulosic material. The lignocellulosic material may be derived from a wood material.

In one aspect, the present invention relates to a method and/or a system to pretreat biomass to be used in a biorefinery to produce a biofuel or biochemical using a fermentation process, said method or system comprising the steps of:
(a) preparing the biomass into prepared biomass;
(b) pre-treating the prepared biomass into a pretreated biomass by application of a concentrated acid;
(c) facilitating the production of a fermentation product derived from the biomass to be obtained by separating the pretreated biomass into (1) a liquid component comprising lignin, solvent and acid and (2) a solids component from which glucose can be made available for fermentation into the fermentation product;
(d) recovering recoverable acid from the pre-treated biomass;
(e) producing recovered acid from recoverable acid; and
(f) contributing recovered acid to be used as concentrated acid;

wherein the biomass comprises lignocellulosic material; and

wherein the lignocellulosic material is derived from a wood material.

In one embodiment, the method further comprises the step of treatment of at least one of (a) the liquid component or (b) the pre-treated biomass.

In one embodiment, the treatment comprises adding solvent or water to the pretreated biomass.

In one embodiment, the method further comprises the steps of separation of at least one of (a) the liquid component or (b) pretreated biomass, wherein, preferably, separation comprises at least one of (a) evaporation; (b) distillation; (c) filtration; (d) distillation to recover recoverable acid; (e) separation of the solids component; (f) separation of recoverable acid; (g) filtration to remove lignin and other solids from the liquid component; (h) evaporation to remove water or solvent; or (i) separation of recoverable acid; and wherein recoverable acid is recovered from at least one of the liquid component or the solid component.

In one embodiment, the method further comprises the step of treatment of the solid component; and treatment comprises at least one of (a) at least one wash; (b) dilution of the liquid component; or (c) dissolving lignin and acid with solvent.

In one embodiment, the liquid component is evaporated to recover solvent.

In one embodiment, the treatment comprises of at least one of (a) application of solvent; (b) application of water; (c) addition of water to cause lignin to precipitate; wherein solvent comprises ethanol; wherein application comprises a wash.

In one embodiment, water is added (a) at a ratio from about 1:1 part to 5:1 part water to one part liquid component after evaporation; (b) at a ratio of about 1:1 to about 3:1 water to liquid component after evaporation.

In one embodiment, the concentrated acid comprises phosphoric acid, and/or the liquid component comprises about 10 to 20 percent phosphoric acid, and/or the liquid component comprises about 40 to 60 percent solvent, and/or the liquid component comprises about 0 to 5 percent xylose and about 0 to 2 percent glucose, and/or the liquid component comprises about 1 to 75 percent lignin by weight of the solids.

In one embodiment, the liquid component after lignin removal is subjected to evaporation to remove the water and re-concentrate the phosphoric acid for use as concentrated acid.

In one embodiment, water is added to the liquid component after the solvent is removed.

In one embodiment, the concentrated acid comprises recovered acid and fresh acid; wherein fresh acid comprises acid not previously used to treat biomass; and recovered acid comprises acid recovered from pre-treated biomass.

In one embodiment, the concentrated acid is provided with a concentration in a range of 75 to 100 percent; and the concentrated acid comprises at least between 90 to about 100 percent recovered acid and between 0 to 10 percent fresh acid.

In one embodiment, pre-treating the biomass is performed at operating conditions comprising a temperature in a range of about 20 to about 90 degrees Celsius for a period of time in a range of about 15 minutes to 4 hours.

In one embodiment, the lignocellulosic material comprises 30 to 55 percent cellulose by weight and hemicellulose at about 15 to 40 percent by weight; wherein the lignocellulose material comprises glucan and xylan; and wherein the glucan comprises cellulose; wherein the hemicellulose comprises xylan, and/or the lignocellulosic material comprises at least one of (a) a wood material; or (b) saw dust residue (an agricultural residue); and the wood material comprises at least one of saw dust, saw dust residue; hard wood sawdust consisting of at least oak wood residue from a wood processing facility, oak wood, maple wood, hickory wood, poplar wood, other indigenous hard woods to North America; and agricultural residue comprises at least one of corn stover, wheat straw, oat straw, rice straw, sugar cane bagasse, sugar beet bagasse, empty fruit bunches from palm oil extraction, other agricultural residue, and/or the lignocellulosic material comprises wood residue and comprises cellulose at about 35 to 45 percent cellulose by weight and hemicellulose at about 17 to 24 percent by weight.

In one embodiment, separation is performed in a liquid-solid separator unit comprising at least one of (a) a plate and frame filter press or (b) a basket centrifuge.

### BRIEF DESCRIPTION OF THE FIGURES

FIG. 1 is the perspective layout of a bio-refinery comprising a biofuel and/or biochemical production facility according to an exemplary embodiment.

FIG. 2 is a perspective layout of a bio-refinery comprising a biofuel and/or biochemical production facility and a wood processing facility for producing biomass according to an exemplary embodiment.

FIG. 3 is a schematic block diagram of the production facility for producing biofuel and/or biochemical from biomass according to an exemplary embodiment.

FIG. 4 is a schematic diagram of a system for receiving and preparing the biomass for biofuel and/or biochemical production facility according to an exemplary embodiment.

FIG. 5 is a schematic block diagram for the production of biofuel and/or biochemical from biomass.

FIGS. 6A, 6B and 6C are schematic block diagrams of systems for treating and processing all the components exiting from the biofuel and/or biochemical production facility according to an exemplary embodiment.

FIG. 7 is a schematic block diagram of the process flow of the systems for the production of biofuel and/or biochemical from biomass according to an exemplary embodiment.

FIG. 8A is a schematic block diagram representing the system used for the preparation, pre-treatment and separation of biomass according to an exemplary embodiment.

FIG. 8B is a schematic block diagram of the system used for pre-treating and separating the biomass according to an exemplary embodiment.

FIG. 9A is a schematic diagram of an apparatus used for the preparation, pretreatment and separation of biomass according to the exemplary embodiment.

FIG. 9B is a schematic diagram of an exemplary embodiment of an apparatus used for the pretreatment and separation of biomass.

FIG. 10 is a schematic diagram of an exemplary embodiment of an apparatus used for treatment of Black Liquor to recover ethanol according to an exemplary embodiment.

FIG. 11 is a schematic diagram of an exemplary embodiment of an apparatus used for recovering Lignin from Crude Acid according to an exemplary embodiment.

FIG. 12 is a schematic diagram of an apparatus used for re-concentrating the Dilute Acid stream according to an exemplary embodiment.

FIGS. 13A, 13B, 13C and 13D are line graphs of the operating conditions for the pretreatment system according to an exemplary embodiment.

FIG. 14A is a bar graph representing the pretreatment yield based on acid recovered from each cycle using basket centrifuge according to an exemplary embodiment.

FIG. 14B is a bar graph representing the pretreatment yield based on acid recovered from each cycle using hydraulic press according to an exemplary embodiment.

FIG. 15 is a bar graph depicting the acid present (by weight) in the black liquor in each cycle based on separation, after pretreatment and using basket centrifuge according to an exemplary embodiment.

FIG. 16 is a bar graph depicting the acid present (by weight) in the water washes in each cycle based on separation, after pretreatment and using basket centrifuge according to an exemplary embodiment.

FIG. 17 is a bar graph showing the amount of acid recovered from black liquor in each cycle using basket centrifuge according to an exemplary embodiment.

FIG. 18 is a bar graph showing the amount of ethanol recovered from black liquor in each cycle using basket centrifuge according to an exemplary embodiment.

FIG. 19 is a bar graph showing the glucose yields from pretreated biomass in each cycle using basket centrifuge according to an exemplary embodiment.

FIG. 20 is a bar graph showing overall sugar yields from pretreated biomass based on each acid cycle using basket centrifuge according to an exemplary embodiment.

### TABLES

TABLE 1 is a table of data from concentrated acid pretreatment of lignocellulosic biomass.

TABLE 2 is a table of data from raw biomass composition.

TABLE 3 is a table of data from pretreatment biomass yield (mass basis), acid recovery yield in the 3 major streams, ethanol recovery yield, glucose yields, and an overall process yield are provided.

TABLE 4 is a table of data from hydrolysis filtrate composition and enzyme hydrolysis yield.

TABLE 5 is a table of data from composition of the enzyme hydrolysis filter cake.

### DESCRIPTION

### Introduction

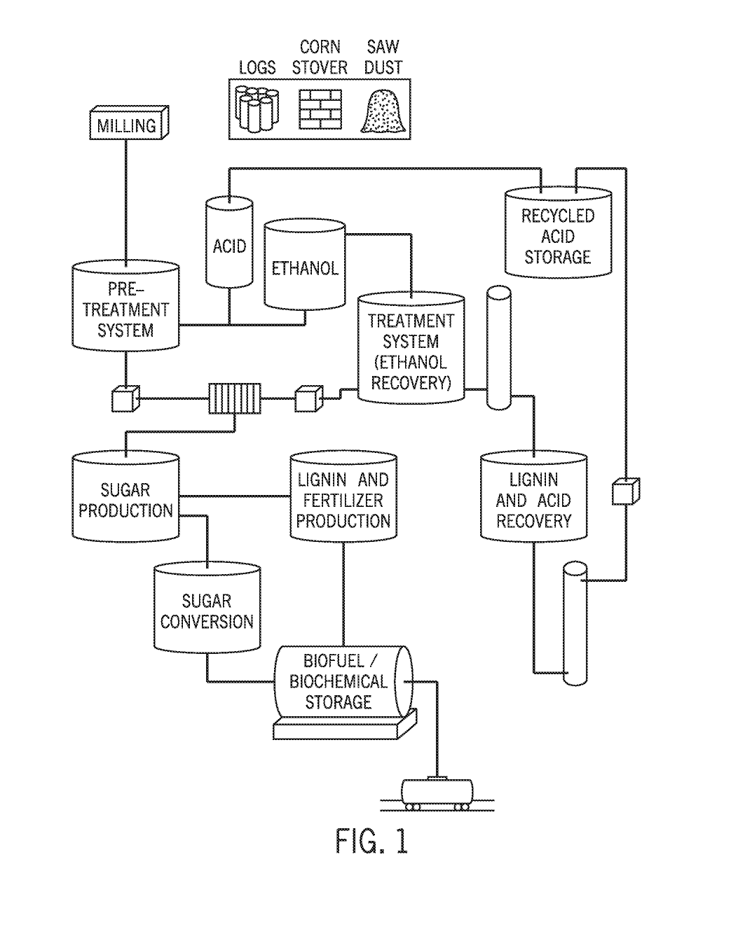

In reference to FIG. 1, a bio-refinery for the production of biofuel and/or biochemical from biomass is shown schematically according to an exemplary embodiment.

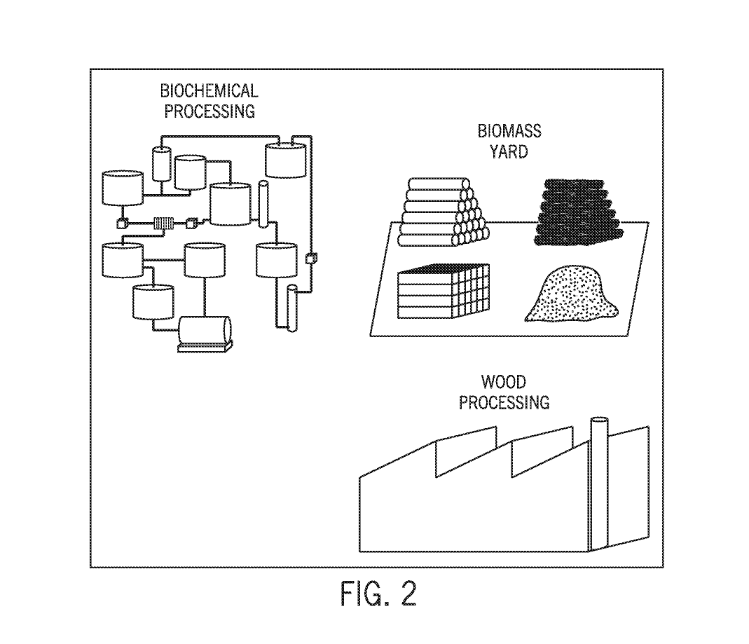

The exemplary embodiment is configured to produce biofuel and/or biochemical from biomass in the form of a lignocellulosic feedstock such as saw dust from a wood processor (e.g. lumber mill, hard wood flooring manufacturer, etc.). See also FIGS. 1 and 2. The lignocellulosic material may comprise cellulose, hemicellulose, lignin, minerals, ash, and minor components including acetic acid.

As shown in FIG. 1, the bio-refinery comprises of an area where the biomass is stored and prepared to be supplied to the biofuel and/or biochemical production facility. As shown schematically according to an exemplary embodiment, the production facility comprises apparatus/equipment for the preparation, pretreatment, separation and treatment of the biomass into treated biomass suitable for bioprocessing (e.g. enzymatic hydrolysis and fermentation). The facility comprises of a distillation system in which acid and ethanol are recovered. As shown in FIG. 1, the bio-refinery may also comprise a lignin and fertilizer production system. The lignin and fertilizer production system may be configured to treat, process and recover components from the residue (e.g. solid cake) after the biofuel production process (e.g. using biochemical reactors).

As shown in FIG. 2, the exemplary embodiment represents a bio-refinery for biofuel and/or biochemical production co-located with a wood processing facility (which processes wood to produce biomass available as feedstock for bioprocessing). As indicated in FIG. 2, by co-locating the two production facilities, certain plant systems can be shared, for example, systems for dehydration, storage of biomass, transportation, utilities, plant management and control systems, and other systems. The processed wood from the wood processing facility is supplied to the bio-refinery for biofuel and/or biochemical production as feedstock. Utility resources such as electricity and water may be used for either or both co-located facilities.

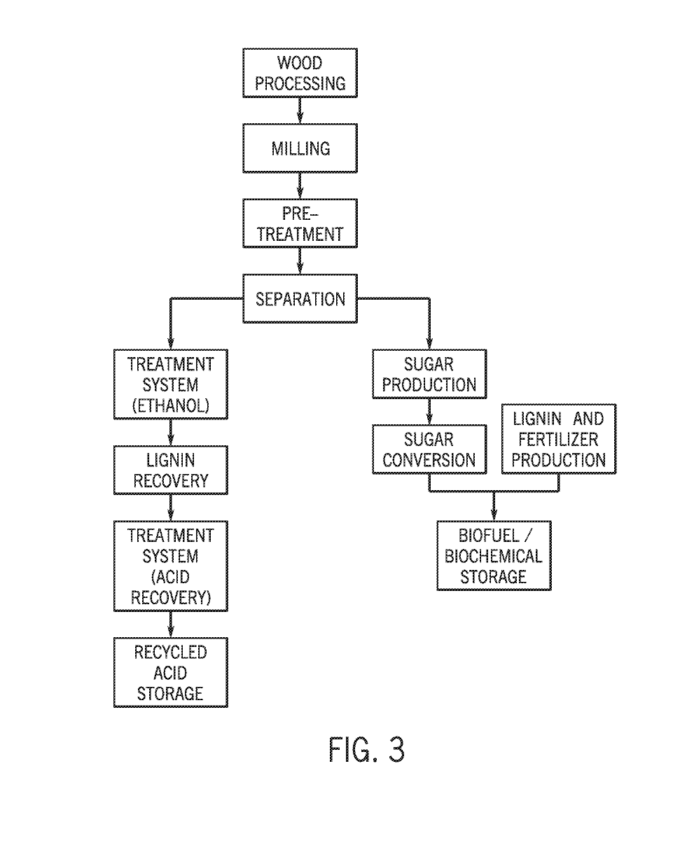

Referring to FIG. 3, a schematic block diagram which represents a bio-refinery for biofuel and/or biochemical production. The wood processing facility supplies biomass as raw material or feedstock for the bio-refinery. The supplied biomass is processed through a milling system configured for preparing biomass. As shown schematically according to an exemplary embodiment, the prepared biomass is supplied to a pretreatment system which is followed by a separation system. The liquid fraction from the separation system is supplied to a treatment system for acid and ethanol recovery. See FIG. 3. The solids from the separation system are supplied to the enzymatic hydrolysis and fermentation facility for sugar and biofuel production respectively. As shown in FIG. 3, the solids from the enzymatic hydrolysis are treated and processed to produce biochemical(s).

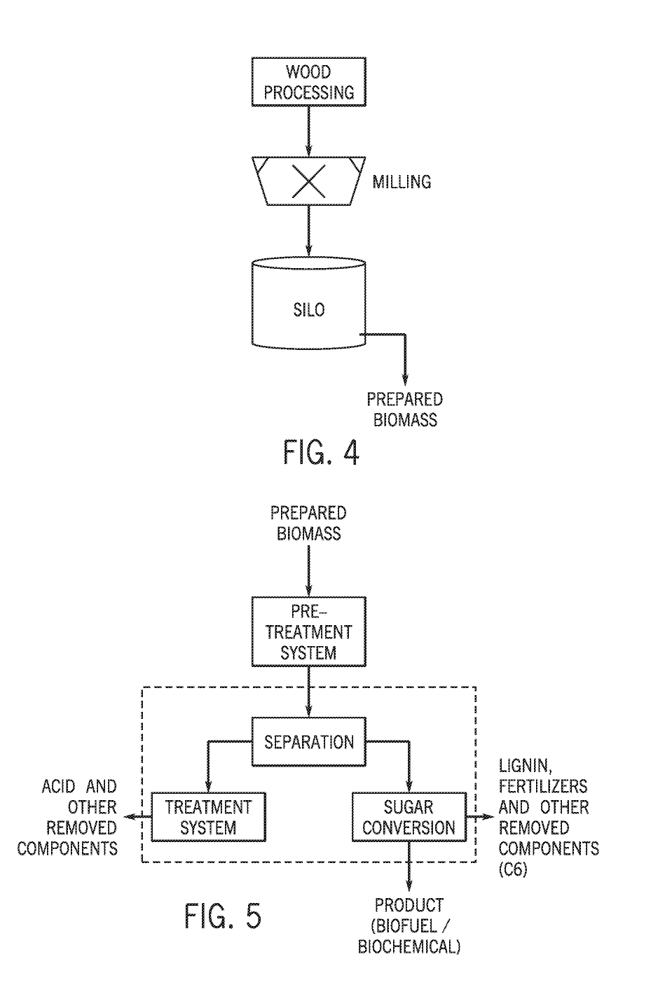

Referring to FIG. 4, a system for the preparation of biomass is shown. As shown schematically according to an exemplary embodiment, the preparation system comprises an apparatus for storing and/or unloading the milled biomass from the wood processing facility. The biomass in the form of sawdust is transported and stored in silos or storage vessels.

Referring to FIG. 5, a schematic block diagram of the cellulosic biofuel production facility is shown schematically according to an exemplary embodiment. The production facility is configured to produce a biofuel or bio-product from biomass comprising lignocellulosic material (e.g. with cellulose and hemicellulose among other content). Cellulose (or glucan polysaccharide) is comprised of hexose (C6) sugars, primarily glucose linked in linear chains. Hemicellulose is branched chain polysaccharide that is comprised of pentose (C5) sugar monomers including xylose and arabinose and smaller amounts of hexose sugar monomers including galactose and mannose; the hemicellulose also contains acetate groups as branches off of the main polymer backbone. According to an exemplary embodiment, the biomass (with cellulose and hemicellulose) is prepared and treated to facilitate access to the hexose (C6) sugars and pentose (C5) sugars for bioprocessing into a biofuel or bio-product. According to the preferred embodiment, the prepared biomass is mixed with acid and is pretreated in a pretreatment system. As shown schematically according to an exemplary embodiment, in the pretreatment system, the biomass is hydrolyzed into a liquid component which mainly comprises of ethanol, water, acid, lignin and sugars; and a solid component which mainly comprises of cellulose from which the C6 sugars can be hydrolyzed. The liquid stream containing the ethanol, water, sugars, lignin and acid; and the solid stream containing the C6 sugars can be treated suitably to obtain the sugars and recover ethanol and acid. The enzymatic hydrolysis and fermentation product can be supplied to a distillation system where the biofuel and/or biochemical is recovered.

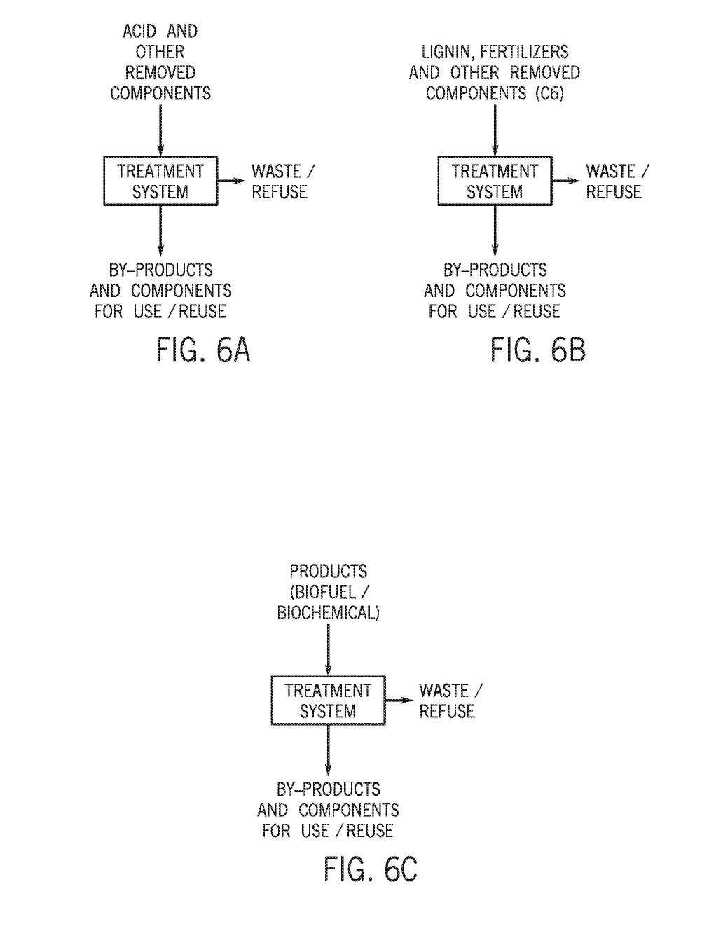

As shown in FIGS. 5 and 6A, the components removed from the treatment of the liquid stream can be further treated or processed to recover the ethanol, phosphoric acid, lignin and organic acid. As shown in FIGS. 5 and 6B, the components removed from the treatment of the C6 stream, such as lignin and other components, can be further treated to obtain clean lignin, which can be used as fuel and fertilizers. As seen in FIGS. 6A, 6B and 6C, all the components removed from the treatment and production of biofuel and/or biochemical from the biomass from either or both the liquid stream and the C6 stream may be processed into bio-products or recovered for use or reuse. As shown in FIG. 6C, biofuel and/or biochemical produced can be treated in a distillation system for use or reuse.

According to the exemplary embodiment, the lignocellulosic biomass (e.g. saw dust) will comprise (by weight) of water and ethanol extractable at about 2-5 percent, cellulose at about 40-50 percent, hemicellulose at about 15-30 percent and lignin at about 20-30 percent.

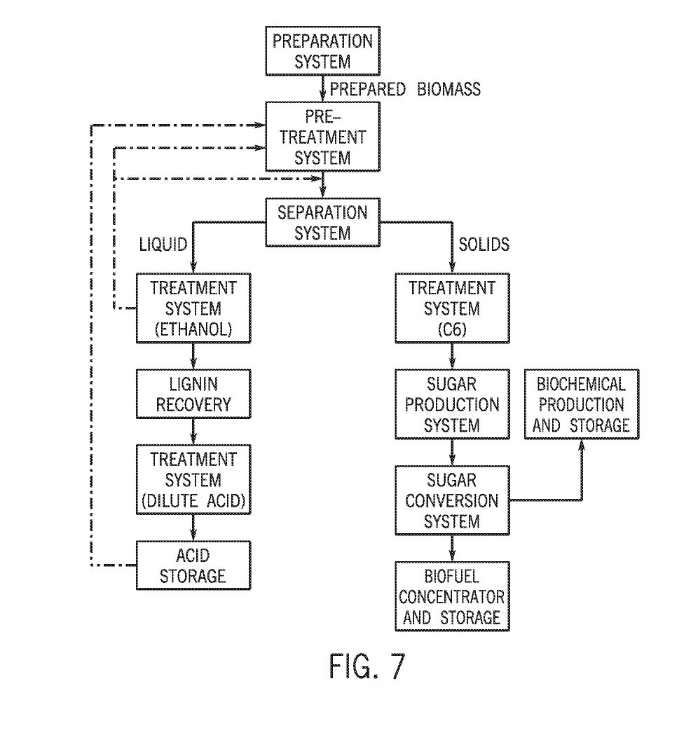

Referring to FIG. 7, the exemplary embodiment of the systems involved in the production of biofuel and/or biochemical are shown. As shown schematically according to an exemplary embodiment, the biomass is pretreated in a pretreatment system and is further separated into a liquid component and solid component.

As shown schematically according to an exemplary embodiment, in the pretreatment system an acid is mixed with the prepared biomass in a known ratio to breakdown the biomass into a liquid component from which acid, lignin and ethanol are recovered and the solids component (C6 stream from which fermentable C6 sugars can be accessed). The solids component after fermentation can be treated to recover lignin and fertilizers for use and/or reuse. According to the preferred embodiment, a known amount of acid is added to the biomass in a reaction vessel under pre-determined operating conditions (i.e. acid concentration, temperature, pressure, time, solids loading, supply of process water or stream, etc.). As shown schematically according to an exemplary embodiment, a mixer is used to agitate to facilitate the breakdown of biomass. As shown schematically according to an exemplary embodiment, an acid such as sulfuric acid, hydrochloric acid, nitric acid, phosphoric acid, acetic acid, etc. can be applied to the biomass. According to a preferred embodiment, treatment by about 85 percent phosphoric acid is used for the process.

As shown schematically according to an exemplary embodiment, the pretreated slurry is quenched with a known amount of ethanol to discontinue the reaction and the mixture undergoes separation in an apparatus into a filtrate or mother liquid and a solid stream (C6 stream from which fermentable C6 sugars can be accessed). The solid component is treated and washed with a known amount of ethanol to remove all the acid, hydrolyzed sugars, organic acids and inorganic material (minerals and ash).

As shown schematically according to an exemplary embodiment, the filtrate from the ethanol washes is combined with the first filtrate or mother liquor to form a composite liquid stream denoted as the black liquor. The black liquor comprises of ethanol, water, dissolved sugars (such as xylose, arabinose, mannose and glucose), lignin, acid and inorganic material (minerals and ash) which are supplied to the distillation system for recovery of ethanol and acid for use and/or reuse. The liquid component (black liquor) may comprise about 1-5 percent solids (i.e. suspended/residual solids such as partially-hydrolyzed hemicellulose, cellulose and lignin). The liquid component (black liquor) will comprise (by weight) of ethanol at about 70-80 percent; phosphoric acid at about 10-15 percent; water at about 1-5 percent and; organic acids and inorganic material (minerals and ash) at about 5-10 percent. The liquid stream (black liquor) is treated in a distillation system to recover ethanol. The stillage or still bottoms (crude acid) contains 1-5 percent of suspended solid which is processed in a lignin recovery system. The crude acid is flocculated with a known flocculant (e.g. flocculant from Nalco included the anionic Nalclear 7763 and Optimer 9825)) and water. The mixture undergoes separation in an apparatus into a liquid component (dilute acid) and a solid component. The solid component is washed with water in the separation apparatus to produce clean lignin. The water washes are combined with dilute acid and the liquid stream is supplied to the distillation unit for acid recovery.

As shown schematically according to an exemplary embodiment, the solid component (C6 stream) comprises of water, acids and solids such as cellulose and hemicellulose from which sugar, such as glucose and xylose, can be made available for fermentation into biofuel and/or biochemical. The solid component is further washed with water in a counter-current fashion to remove any residual hydrolyzed sugars, acids and lignin. On separation, the liquid component comprises of (by weight) expected sugar levels in the black liquor or filtrate are 0-1 percent xylose and glucose, acids (phosphoric acid and organic acids) at about 10-15 percent, water at about 75-85 percent and lignin at about 0.5-1 percent. If the phosphoric acid content of the liquid stream is greater than 5 percent, the liquid stream should be further treated to recover the phosphoric acid for use and/or reuse. According to the exemplary embodiment, the solids component may comprise approximately 20-40 percent solids (by weight) after separation.

During pretreatment, the severity of the operating conditions (temperature, time and concentration) may cause formation of components that are inhibitory to fermentation. For example, under certain operating conditions, dehydration of C5 sugars may result in the increase in concentration of furfural and/or hydroxymethylfurfural (HMF). Pretreatment under certain conditions might result in the release of acetic acid during the break down of the cellulose. The phosphoric acid in the solid component after all the ethanol and water washes must be neutralized or removed or else it might become inhibitory to fermentation. According to the exemplary embodiment, by adjusting the pretreatment conditions (such as temperature, time and concentration), the formation of inhibitors can be reduced or managed. Alternatively the pretreated biomass can be further treated to remove or reduce the level of inhibitors (or other undesirable matter).

As shown schematically according to an exemplary embodiment, the liquid stream from the separation is treated in a distillation system to recover ethanol, phosphoric acid and organic acid for use or reuse. Treatment of the C6 stream (solids component) of the biomass may be performed to make the C6 sugars available for fermentation. According to the preferred embodiment, enzymatic hydrolysis may be performed to access the C6 sugars in the cellulose. Additional treatment may also be performed in an effort to remove lignin and other non-fermentable components in the C6 stream such as residual acid and hydrolyzed sugars.

According to the exemplary embodiment, the C6 sugar stream produced during hydrolysis is fermented to produce C6 fermentation product which is supplied to the distillation system for recovery of biofuel produced from the process. According to the preferred embodiment, a suitable fermenting organism (Novozymes, Lallemand, Leseffre, DSM, National Renewable Energy Lab, DuPont, etc.) will be used in the fermentation system, the selection of the organism is based on types of sugars present in the slurry.

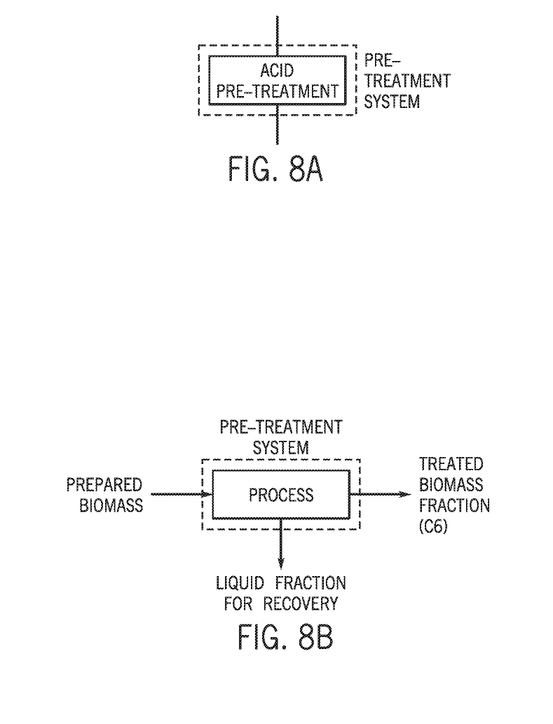

Referring to FIGS. 8A and 8B, the schematic diagram for the pretreatment system is shown. The prepared biomass along with acid and water are supplied to the pretreatment apparatus. As shown schematically according to an exemplary embodiment, the pretreatment vessel is shown as a jacketed reaction vessel with mixing a continuous horizontal reactor containing the prepared biomass, acid and water under predetermined operating conditions such as temperature, reaction time and acid concentration.

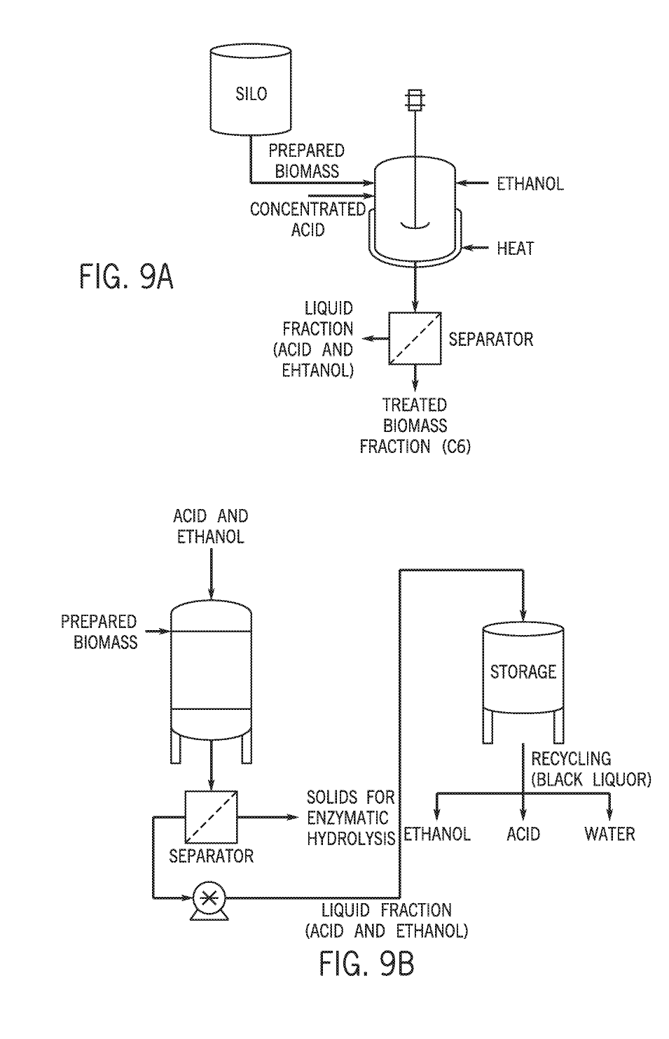

FIGS. 9A and 9B show the apparatus used for carrying out the pretreatment and separation process of lignocellulosic biomass, according to an exemplary embodiment. According to an exemplary embodiment, the biomass is prepared in the mill, milling machine, or grinder using the biomass from the wood processing facility. The prepared biomass along with a known concentration of acid is supplied to at least one jacketed reaction vessel for breaking the cellulosic bonds. Alternatively, pretreatment can be carried out in multiple reaction vessel to improve the yield. As shown in FIG. 9A, the pretreated biomass can be separated using a plate and frame filter or a centrifuge into a liquid component (mother liquor) and a solids component (C6 stream comprising of cellulose, lignin and extractable). As shown in FIG. 9B, the liquid fraction has components such as ethanol and acid which will be recovered for use and/or reuse.

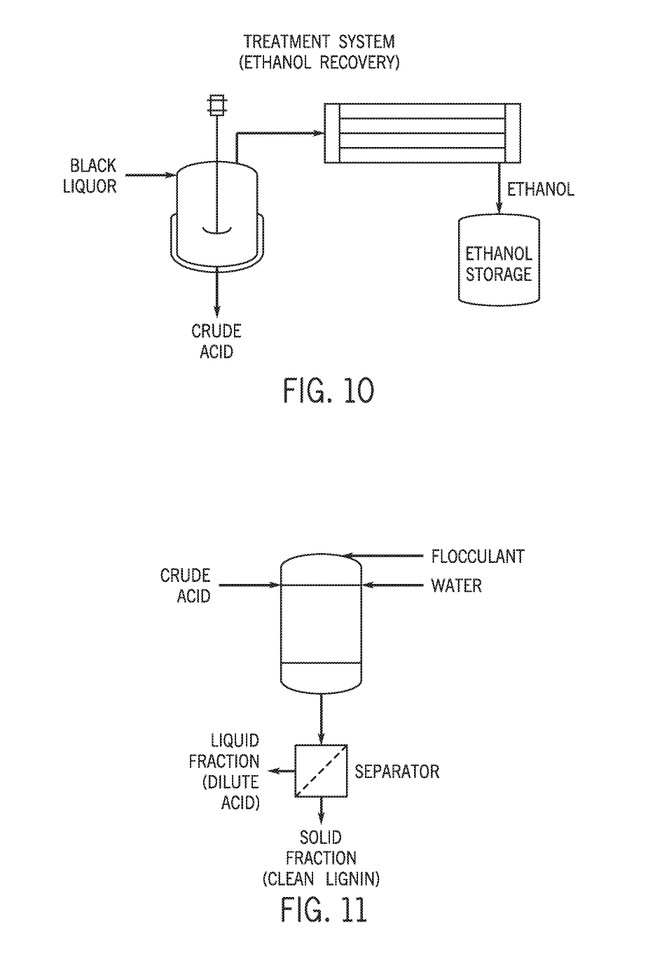

FIG. 10 shows the apparatus used for carrying out ethanol recovery from black liquor according to an exemplary embodiment. According to an exemplary embodiment, a falling film evaporator along with a rectification system can be used. According to a preferred embodiment, a jacketed stirred tank is used for carrying out ethanol and acid recovery. The black liquor is supplied to a jacketed vessel under predetermined conditions (temperature and pressure). The mixer is stirred continuously for better heat transfer. The distillate or overhead product (ethanol) from the vessel is condensed and stored for use and/or reuse. The stillage or still bottoms (crude acid) is further treated to recover lignin and acid for use and/or reuse.

FIG. 11 shows the apparatus used for lignin recovery. The liquid stream (black liquor) is treated in an ethanol recovery system to remove all the ethanol. The crude acid, obtained from the ethanol recovery system, along with a known amount of flocculant and water is supplied to a mixing vessel. The mixing vessel is agitated for carrying out lignin precipitation. The precipitated lignin mixture is supplied to a separation apparatus (plate and frame filter or a centrifuge) from which we obtain a liquid stream (dilute acid) and a solid stream (crude lignin). The crude lignin is washed with water to remove all the acid, hydrolyzed sugars and inorganic material and is termed as clean lignin. The liquid stream obtained from the washes is combined with the dilute acid stream for acid recovery.

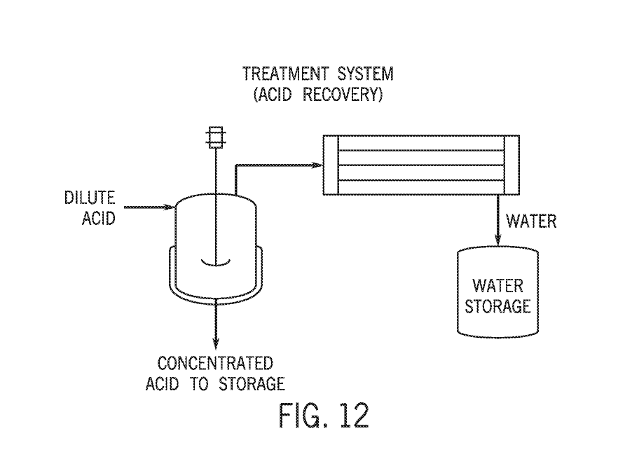

FIG. 12 shows the apparatus for recovering acid. The dilute acid stream obtained after lignin recovery is supplied to a distillation apparatus for acid recovery. The dilute acid is supplied to a jacketed vessel under predetermined operating conditions (temperature and pressure). The mixture is agitated for better heat transfer. The distillate (water) is stored in a vessel which can be treated further for use and/or reuse. The still bottoms or stillage (concentrated acid) is stored in a storage vessel. The recovered acid is reused for carrying out pretreatment of the biomass.

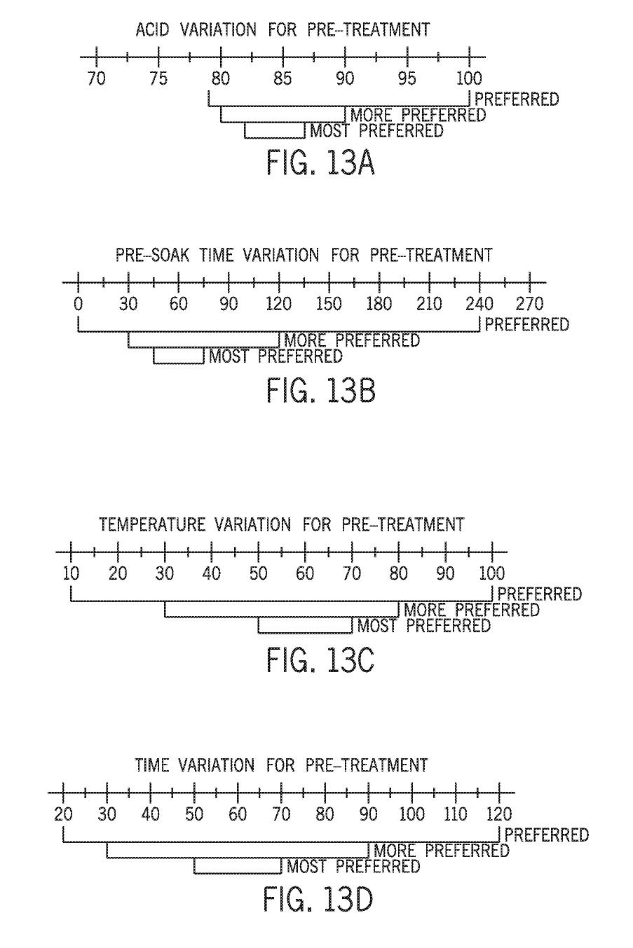

FIGS. 13A through 13D show operating conditions for the pretreatment of biomass according to an exemplary embodiment. Each of FIGS. 13A to 13D (each diagram) shows a generally acceptable operating range (outer/wide range), a preferred operating range (middle range), and a particularly preferred operating range (inner/narrow range) for operating parameter to carry out the pretreatment of the biomass.

FIG. 13A shows the acid concentration (phosphoric acid in water) for operation of the pretreatment system (in percent, by weight). According to a exemplary embodiment, the acid concentration is from about 79-100 percent phosphoric acid in the pretreatment solution. According to a preferred embodiment, the acid concentration is from about 80-90 percent. According to a particularly preferred embodiment, the acid concentration is from about 82-87 percent.

FIG. 13B shows the presoak time for carrying out the pre-treatment process. According to the exemplary embodiment, the presoak time is from about 0 to 240 minutes. According to the preferred embodiment, the operating time is from about 30 to 120 minutes. According to the particularly preferred embodiment, the pretreatment time is from about 45 to 75 minutes.

FIG. 13C shows the temperature ranges for operating the pretreatment system. According to the exemplary embodiment, the operating temperature range for the pretreatment process is about 10 to about 100 degrees Celsius. According to the preferred embodiment, the operating temperature range for the pretreatment process is about 30 to about 80 degrees Celsius. According to the particularly preferred embodiment, the operating temperature range for the pretreatment process is about 50 to about 70 degrees Celsius.

FIG. 13D shows the reaction time for carrying out the pretreatment process. According to the exemplary embodiment, the pretreatment time is from about 20 to 120 minutes. According to the preferred embodiment, the operating time is from about 30 to 90 minutes. According to the particularly preferred embodiment, the pretreatment time is from about 50 to 70 minutes.

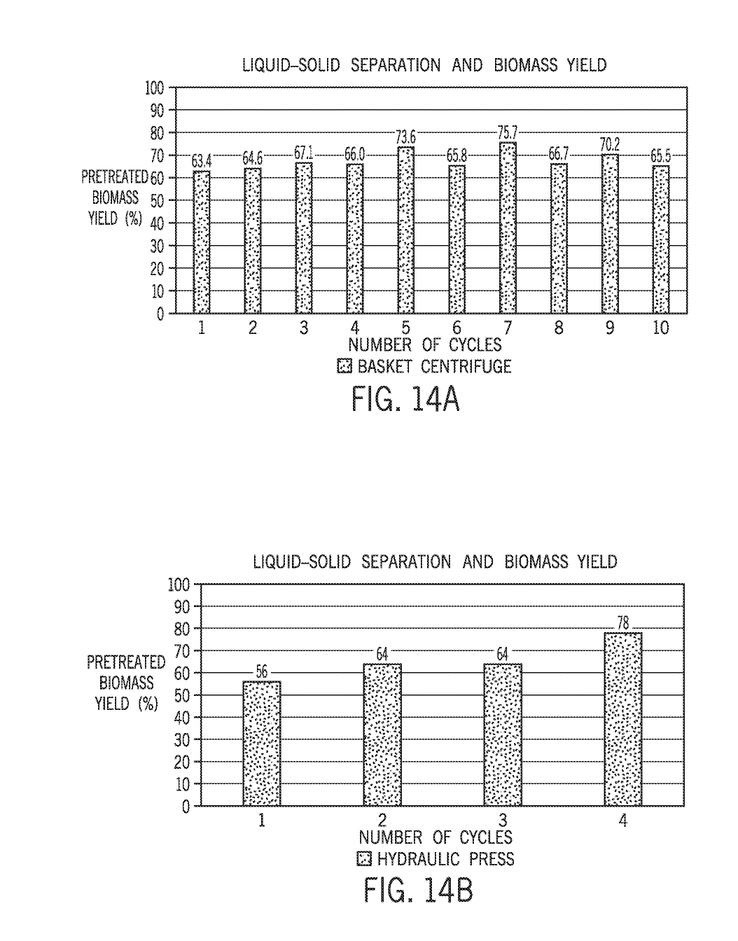

Referring to FIGS. 14A and 14B, according to the preferred embodiment, a known amount of acid is added to the biomass in a reaction vessel under predetermined operating conditions (i.e. acid concentration, temperature, pressure, time, solids loading, supply of process water or stream, etc.).

The pretreated slurry is quenched with a known amount of ethanol to discontinue the reaction and the mixture undergoes separation in an apparatus to form a filtrate (e.g. mother liquor) and solid component (C6 stream from which fermentable C6 sugars can be accessed). The solid component is washed with a known amount of ethanol to remove nearly all the acid, hydrolyzed sugars, organic acids and inorganic material (minerals and ash). The filtrate from the ethanol washes is combined with the original filtrate or mother liquor forming a combined filtrate of black liquor. The black liquor comprises of ethanol, water, dissolved sugars (such as xylose, arabinose, mannose and glucose), organic acids and inorganic material (minerals and ash). The combined filtrate or black liquor may comprise 1-5 percent solids (i.e. suspended/residual solids such as partially-hydrolyzed hemicellulose, cellulose and lignin). The combined filtrate or black liquor will comprise (by weight) of ethanol at about 70-80 percent; phosphoric acid at about 10-15 percent; water at about 1-5 percent and; organic acids and inorganic material (minerals and ash) at about 5-10 percent.

The combined filtrate or black liquor is supplied to a distillation apparatus to remove and recover the ethanol. The lignin present in the liquid stream (crude acid), after ethanol removal, is precipitated using a known amount of flocculant and water. The mixture undergoes separation in an apparatus into a solid component (e.g. lignin) and a filtrate (e.g. dilute acid) from which acid was re-concentrated and recycled using a distillation apparatus.

The first separation technique involves a basket centrifuge (BC). The acid recycled using the BC was efficient for the pretreatment of biomass for up to 10 cycles. The pretreated biomass solids yield using the BC for acid recovery has an average recovery rate of about 68 percent.

An alternative to the BC is the hydraulic press which can be used as a solid-liquid separation technique, the acid strength of mother liquor (ethanol quench) collected from cycle 1 through 4 is in the range 29-34 percent and the liquid component from the ethanol washes is approximately 4 percent. The total acid present in black liquor (mother liquor and the liquid component from ethanol washes) (by weight) from cycle 1 through 4 are about 98, 111, 94, and 96 percent with an average of about 100 percent. The hydraulic press work was discontinued as the sugar production during enzyme hydrolysis was less than that of the BC treated materials. The amount of acid presented is given based on 85 percent strength.

As per FIG. 14B, the second separation technique involves a hydraulic press (HP). The acid recycled using the hydraulic press was efficient for the pretreatment of biomass for up to 3 cycles and the recycled acid used for cycle 4 pretreatment was not efficient. The pretreated biomass yields, using a hydraulic press, were about 56, 64, 64, and 78 percent with an average yield of about 66 percent. The pretreated biomass yield in cycle 4 is higher due to phosphoric acid being saturated with dissolved sugars and organic acids.

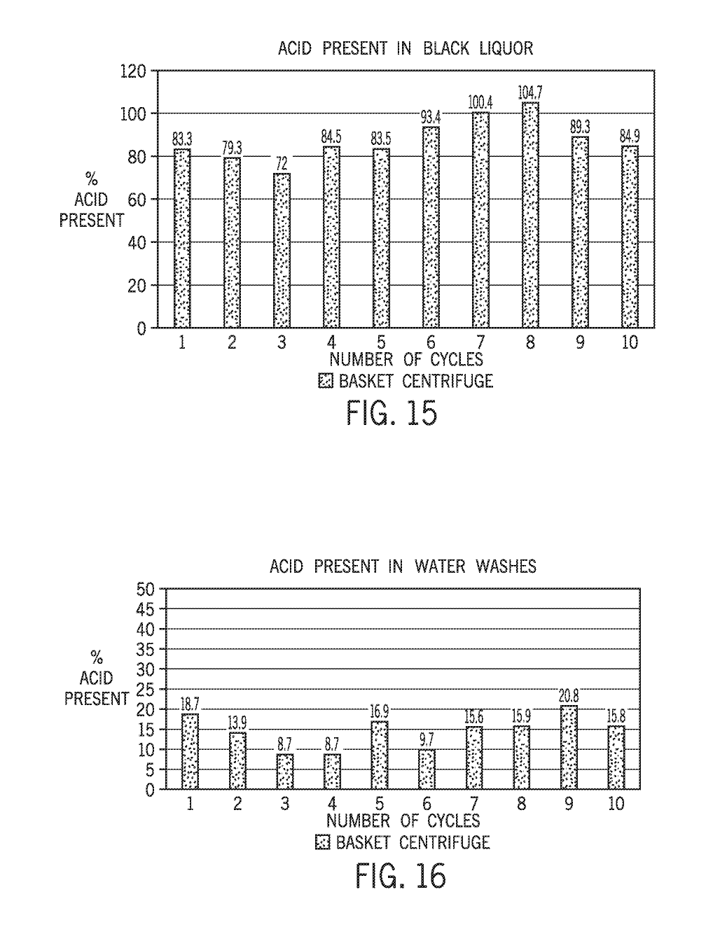

FIG. 15 shows the amount of acid present in the black liquor. The amount of acid present is determined through titrations using 0.2M sodium hydroxide solution (NaOH) as titrant.

When the basket centrifuge is used as a solid-liquid separation technique, the acid strength of mother liquor (ethanol quench) collected from cycle 1 through 10 is in the range of about 25-31 percent and the liquid component from the ethanol washes is in the range 1.4-8.8 percent. The total acid present (by weight) in black liquor from cycle 1 through 10 are 83, 79, 72, 85, 84, 93, 100, 105, 89, and 85 percent with an average 88 percent. The amount of acid presented is based on 85 percent strength.

FIG. 16 shows the amount of acid present in water washes. The amount of acid is determined through titrations using 0.2 M sodium hydroxide solution (NaOH) as titrant over the water wash samples.

When the basket centrifuge is used as a solid-liquid separation technique, total acid present (by weight) in cycle 1 through 10 are 19, 14, 9, 9, 17, 10, 16, 16, 21, and 16 percent with an average 14 percent. The amount of acid presented is based on 85 percent strength. Lower acid content in water washes implies minimum acid loss and higher acid recovery for the process. The acid present in the water washes will be converted to bio-chemicals.

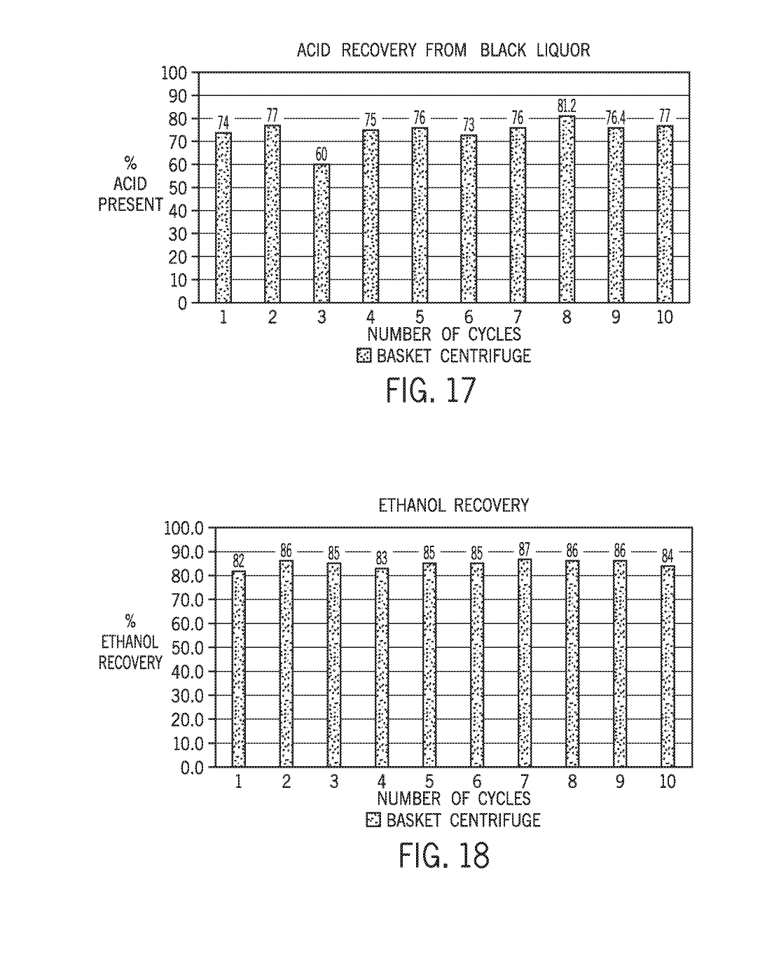

FIG. 17 shows the amount of acid recovered from black liquor. The amount of acid (by weight) recovered using basket centrifuge from cycle 1 through 10 are 74, 77, 60, 75, 76, 73, 76, 81, 76, and 77 percent with an average of about 75 percent. All the acid recovery yields presented were with 85 percent acid strength. The total recovered acid from each cycle is supplied to the next pretreatment cycle and the loss was compensated by adding fresh 85 percent phosphoric acid. The acid recovery yields were lower as compared to the amount of acid present in black liquor due to physical and process losses (acid transfer from evaporation process, lignin precipitation, and samples collected for analysis). The solid content of the material from the basket centrifuge is 25-30 percent, indicating that the solids retain some amount of acid and ethanol causing acid loss into the water washes.

The acid recovered using basket centrifuge retained is effective through cycle 10. Further experiments were deemed unnecessary as a steady trend of sugars yield was observed from the subsequent enzyme hydrolysis process. As the acid recovery is not 100 percent, the acid loss is compensated with fresh phosphoric acid for each pretreatment cycle was 25 percent in case of basket centrifuge. In case of basket centrifuge, the higher amount of fresh phosphoric acid may be responsible for better acid efficiency for next pretreatment cycle.

FIG. 18 shows the amount of ethanol extracted using the basket centrifuge. The amount of ethanol (by weight) recovered using basket centrifuge from cycle 1 through 10 are 82, 86, 85, 83, 85, 85, 87, 86, 86, and 84 percent with an average of about 85 percent. The solid content using basket centrifuge is 25-30 percent since it retains excess acid and ethanol in the pretreated biomass causing acid loss into water washes.

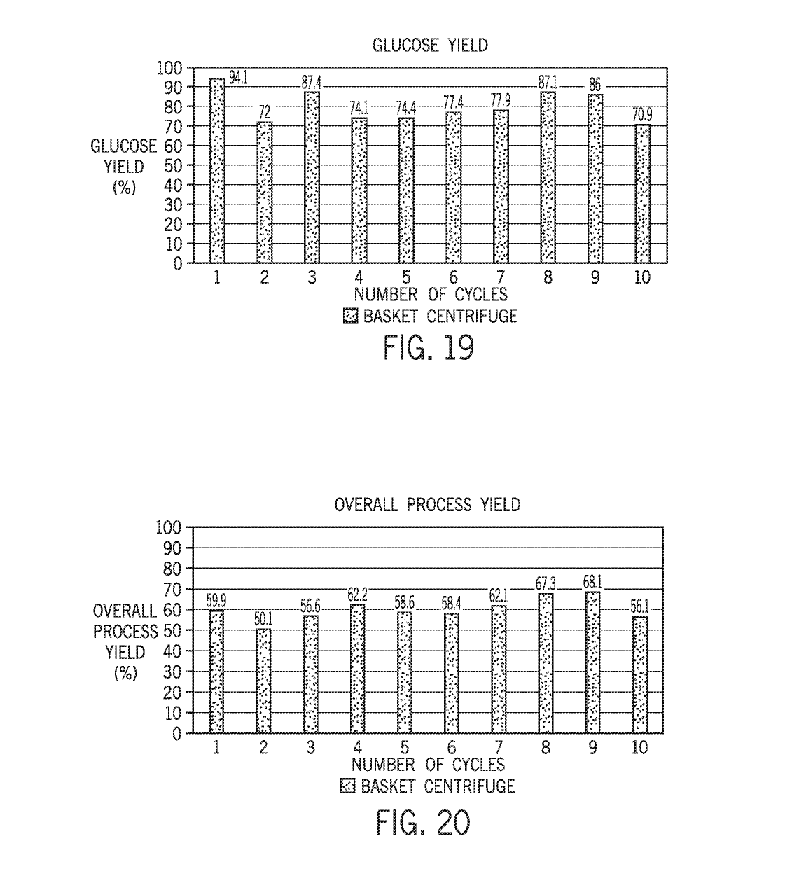

FIG. 19 shows the glucose yields using the basket centrifuge as a separation technique. The biomass is supplied to a pretreatment apparatus along with a known amount of acid. The material is quenched with a known amount of ethanol which is supplied to a separation apparatus. The solid component is washed with ethanol and water in a predetermined ratio. The solids are supplied to the enzymatic hydrolysis for producing sugars (glucose and xylose) which will be fermented to produce biofuel and/or bio-chemicals. The glucose yields using basket centrifuge from cycle 1 through 10 are 94, 72, 87, 74, 74, 77, 78, 87, 86, and 71 percent, with an average 80 percent. When the basket centrifuge is used for solid-liquid separation a lower force or pressure is exerted by the centrifuge causing more liquid phase to be retained in the solids. Since the basket centrifuge materials have higher liquid or moisture content, they are pressed once in the hydraulic press to approximately 45 percent solids (55 percent moisture). These solids are then passed on the enzyme hydrolysis process for sugar generation via enzymes.

FIG. 20 shows the overall sugar yields after the pretreated biomass is supplied to enzymatic hydrolysis for sugar generation. When the overall sugar yields when we use the basket centrifuge from cycle 1 through 10 are 60, 50, 57, 62, 59, 58, 62, 67, 68, and 56 percent with an average 58 percent. The decrease in overall sugar yields is due to loss of hemicellulose sugars in to black liquor and water washes.

### EXAMPLES

A series of examples were conducted according to an exemplary embodiment of the system in an effort to evaluate the recyclability of acid stream recovered by washing the acid from the pretreated biomass. The examples start with the pretreatment process and conditions creating a baseline process. The acid is recovered by liquid-solid separation creating a filtrate (e.g. the mother liquor). Once the mother liquor or filtrate is removed the residual acid is removed by washing with additional solvent (e.g. ethanol). This washing process can be carried out co-currently or counter currently. Counter current washing is preferred as it requires less solvent to do the washing creating a much smaller filtrate stream that eventually requires evaporation. Both washing approaches may provide generally equivalent acid recoveries.

### Example 1

### Pretreatment Process

The system and method was used in Example 1 to determine the pretreatment process.

TABLE 1 provides information for the treatment or pretreatment process including comments regarding how the process is carried out to reproduce the work.

TABLE 1
Concentrated acid pretreatment of lignocellulosic biomass

| Component | Mass (Kg) | Comments |
|---|---|---|
| Saw dust (American Wood | 37 (as delivered) | The moisture in the wood (e.g. 5 to 15 |
| Fiber) | | percent as delivered) is accounted for in |
| | | the calculations for acid addition. TABLE |
| | | 2 provides the composition of the raw |
| | | wood. |
| Phosphoric acid (83 percent | 185 | The saw dust and acid are mixed together |
| via titration from Potash | | followed by a 60 minute presoak or |
| Corp.) | | chemical impregnation period. At the end |
| | | of the 60 minute impregnation, the material |
| | | is heated to 60 degrees Celsius and |
| | | maintained there for 60 minutes to “cook” |
| | | the wood. |
| Ethanol (95 percent - | 370 | The ethanol is added to quench the acid |
| Brentag) | 555 | treated saw dust and to cause the |
| | | precipitation of the cellulose. The |
| | | quenched material is filtered and the |
| | | filtrate retained. The solid cake remains in |
| | | the filtration system where it is washed |
| | | with 3 solvent washes in a counter current |
| | | manner with the additional 555 kg of |
| | | solvent. |
| Water (city water) | 2775 | Once the solvent washing is complete, the |
| | | solvent is forced out of the sold cake by |
| | | washing with water until the water has less |
| | | than 0.2 percent acid by weight in the |
| | | water. |
| Mixer (Charles Ross and | | 945 liter tank with anchor style mixing |
| Sons) | | blades on the center shaft and disperser |
| | | blade on the offset shaft. |
| Plate and Frame Filter (Ertyl | | 24 plates with filtration media optimized |
| Alsop) | | for the filtration of the treated sawdust |
| | | material. |

The expected outcome of the experiment was the pretreatment of the lignocellulosic biomass in way that allows for the recovery and recycle of the acid, useful pretreatment of the cellulose allowing for the production of glucose from the cellulose at yields of about 80 to 100 percent and recovery of the acid and solvent at levels greater than about 95 percent. The filtrate or mother liquor from the first separation after the quench process is combined with the filtrates from the counter current washes forming a combined filtrate or black liquor. From the acid recycling process, the combined filtrate becomes cycle 1. The black liquor or combined filtrate is evaporated to recover the ethanol for recycling (e.g. ethanol is the distillate and the acid and other residues are the still bottoms). Water is added to the still bottoms along with an anionic flocculent (e.g. flocculent from Nalco included the anionic Nalclear 7763 and Optimer 9825) causing the lignin and other similar components to precipitate. As indicated additional flocculants that will work about the same as the Nalco products. The lignin is recovered by filtration and washing with additional water forming a lignin cake in the filtration system. The filtrate contains the acid in a diluted form creating a dilute acid stream. The acid is recovered from the dilute acid stream and concentrated to about 75 to 100 percent concentration by evaporation (e.g. water becomes the distillate and the now concentrated acid becomes the still bottoms or stillage). The amount or mass of the original acid that has been recovered and recycled will be about 85 percent to about 100 percent.

The results from Example 1 provided in TABLES 3, 4, and 5 below show the yields from the process steps to convert lignocellulosic biomass to a fermentation product while recovering and recycling the process chemicals creating a more technically and economically feasible process enabling a commercial scale biorefinery using the process approach.

TABLE 2
Raw biomass composition

| | Ash | Water | | | | | | | | | | | | | |
|---|---|---|---|---|---|---|---|---|---|---|---|---|---|---|---|
| Raw | (ext- | ex- | | Ash | Lignin | Lignin | | | | | Man- | Su- | Uronic | | Mass |
| Biomass | free) | tractable | EtOH | (soil) | (AIL) | (ASL) | Glucan | Xylan | Galactan | Arabinan | nan | crose | acid | Acetyl | closure |
| 060614-A | 1.0% | 3.3% | 1.4% | 0.6% | 24.5% | 0.3% | 42.0% | 17.2% | 0.0% | 0.0% | 4.2% | 0.0% | 1.1% | 4.3% | 99.9% |
| 060614-B | 0.9% | 2.7% | 1.0% | 0.5% | 23.7% | 0.3% | 43.4% | 16.6% | 0.0% | 0.0% | 2.1% | 0.0% | 1.5% | 4.9% | 99.7% |
| 060614-C | 0.9% | 3.1% | 1.2% | 0.3% | 24.2% | 0.3% | 42.2% | 17.4% | 0.0% | 0.0% | 4.3% | 0.0% | 1.1% | 4.3% | 99.6% |
| Average | 0.9% | 3.1% | 1.2% | 0.5% | 24.1% | 0.3% | 42.5% | 17.1% | 0.0% | 0.0% | 4.3% | 0.0% | 1.3% | 4.5% | 99.7% |

TABLE 3
Pretreatment Biomass yield (mass basis), acid recovery yield in the 3 major streams,
ethanol recovery yield, glucose yields, and an overall process yield are provided

| | Cycle Number | | | | | | | | | | |
|---|---|---|---|---|---|---|---|---|---|---|---|
| | 1 | 2 | 3 | 4 | 5 | 6 | 7 | 8 | 9 | 10 | Mean |
| Pretreatment Mass Yield | 63.4 | 64.6 | 67.1 | 66.0 | 73.6 | 65.8 | 75.7 | 66.7 | 70.2 | 65.5 | 68.1 |
| (%) | | | | | | | | | | | |
| (Basket Centrifuge) | | | | | | | | | | | |
| Pretreatment Mass Yield | 56.2 | 64.0 | 64.0 | 78.0 | — | — | — | — | — | — | 65.6 |
| (%) | | | | | | | | | | | |
| (Hydraulic Press) | | | | | | | | | | | |
| Acid in Black Liquor | 83.3 | 79.3 | 72 | 84.5 | 83.5 | 93.4 | 100.4 | 104.7 | 89.3 | 84.9 | 87.6 |
| (%) | | | | | | | | | | | |
| (Basket Centrifuge) | | | | | | | | | | | |
| Acid in Water Wash | 18.7 | 13.9 | 8.7 | 8.7 | 16.9 | 9.7 | 15.6 | 15.9 | 20.8 | 15.8 | 13.5 |
| (%) | | | | | | | | | | | |
| (Basket Centrifuge) | | | | | | | | | | | |
| Acid Recovered | 74 | 77 | 60 | 75 | 76 | 73 | 76 | 81.2 | 76.4 | 77 | 74.6 |
| (%) | | | | | | | | | | | |
| (Basket Centrifuge) | | | | | | | | | | | |
| Ethanol Recovered | 82.0 | 86.0 | 85.0 | 83.0 | 85.0 | 85.1 | 87.0 | 86.0 | 86.0 | 84.0 | 84.9 |
| (%) | | | | | | | | | | | |
| (Basket Centrifuge) | | | | | | | | | | | |
| Glucose Yields | 94.1 | 72 | 87.4 | 74.1 | 74.4 | 77.4 | 77.9 | 87.1 | 86 | 70.9 | 79.9 |
| (%) | | | | | | | | | | | |
| (Basket Centrifuge) | | | | | | | | | | | |
| Overall Process Yield | 59.9 | 50.1 | 56.6 | 62.2 | 58.6 | 58.4 | 62.1 | 67.3 | 68.1 | 56.1 | 57.6 |
| (%) | | | | | | | | | | | |
| (Basket Centrifuge) | | | | | | | | | | | |

TABLE 4
Hydrolysis filtrate composition and enzyme hydrolysis yield

| | | | | | | | | HYD | HYD HC | HYD |
|---|---|---|---|---|---|---|---|---|---|---|
| Glucose | Cellobiose | Glucose | Xylose | Galactose | Arabinose | Mannose | Density | Glucose | Sugars | Process |
| Stream | (g/l) | (g/l) | (g/l) | (g/l) | (g/l) | (g/l) | (g/mL) | Yield (%) | Yield (%) | Yield (%) |
| Cycle | 3.61 | 157.85 | 5.005 | 1.06 | 0 | 1.275 | 1.05 | 94.1% | 6.4% | 59.9% |
| Number 1 | | | | | | | | | | |
| Cycle | 8.805 | 105.17 | 2.46 | 1.41 | 0 | 5.35 | 1.05 | 72.0% | 9.3% | 50.1% |
| Number 2 | | | | | | | | | | |
| Cycle | 11.145 | 128.94 | 0 | 0.225 | 0 | 0 | 1.1 | 87.4% | 0.2% | 56.6% |
| Number 3 | | | | | | | | | | |
| Cycle | 7.18 | 132.76 | 0 | 3.125 | 0 | 10.91 | 1.07 | 74.1% | 0.0% | 62.2% |
| Number 4 | | | | | | | | | | |
| Cycle | 8.09 | 119.895 | 0 | 0 | 0 | 2.22 | 1.072 | 74.4% | 0.0% | 58.6% |
| Number 5 | | | | | | | | | | |
| Cycle | 4.505 | 139.545 | 0 | 0.72 | 0 | 0.485 | 1.073 | 77.4% | 1.8% | 58.4% |
| Number 6 | | | | | | | | | | |
| Cycle | 7.78 | 114.845 | 3.06 | 0 | 0 | 1.795 | 1.053 | 77.9% | 8.4% | 62.1% |
| Number 7 | | | | | | | | | | |
| Cycle | 11.225 | 148.98 | 0 | 0 | 0 | 0.14 | 1.077 | 87.1% | 0.2% | 67.3% |
| Number 8 | | | | | | | | | | |
| Cycle | 8.42 | 133.93 | 0 | 1.76 | 0 | 3.63 | 1.032 | 86.0% | 8.8% | 68.1% |
| Number 9 | | | | | | | | | | |
| Cycle | 6.855 | 125.52 | 4.32 | 0 | 0 | 1.64 | 1.095 | 70.9% | 8.6% | 56.1% |
| Number 10 | | | | | | | | | | |

TABLE 5
Composition of the enzyme hydrolysis filter cake

| Sample | Ash | | | | | | | | | | |
|---|---|---|---|---|---|---|---|---|---|---|---|
| (Pretreated | (ext- | Lignin | Lignin | | | | | | Glucuronic | | |
| Biomass) | free) | (AIL) | (ASL) | Glucan | Xylan | Galactan | Arabinan | Mannan | acid | Acetyl | Mass closure |
| Cycle | 2.3% | 28.5% | 0.0% | 53.7% | 3.9% | 0.0% | 0.0% | 0.0% | 3.2% | 1.0% | 92.7% |
| Number 1 | | | | | | | | | | | |
| Cycle | 0.8% | 33.7% | 0.3% | 51.2% | 7.7% | 0.0% | 0.0% | 3.1% | 0.9% | 1.2% | 98.9% |
| Number 5 | | | | | | | | | | | |
| Cycle | 1.1% | 31.5% | 0.3% | 56.4% | 8.6% | 0.0% | 0.0% | 0.0% | 0.3% | 0.6% | 98.8% |
| Number 8 | | | | | | | | | | | |

### Example 2

### Acid Recovery Process

The system and method was used in Example 2 to determine the acid recovery process using the method and process described in Example 1. See also Example 4 (effect on sugars production). The recovered and concentrated acid from Cycle 1 was recycled into the process and used to conduct another treatment or pretreatment of the lignocellulosic biomass. The pretreated material was separated as in Example 1 followed by the recovery of ethanol, precipitation of lignin, and the concentration of the acid. The solid cake from the separation process was processed further to make sugar in the enzyme hydrolysis or sugar production process step. The acid was once again recovered as in Example 1 (e.g. evaporation of the solvent, precipitation of the lignin, and evaporation of the water making the Cycle 2 concentrated acid). The process step once again recovers and recycles about 85 percent to about 100 percent of the acid put into the treatment or pretreatment process step. The recovered and recycled acid seemed to have no significant impact on sugar production process yields. The data relating to acid recovery generated while preparing the Cycle 2 acid is provided above in TABLES 3, 4, and 5.

### Example 3

### Reuse of Recovered Acid for Biomass Pretreatment (Hydraulic Press Vs Basket Centrifuge)

The system and method was used in Example 3 to evaluate re-use of recovered acid for biomass pretreatment (hydraulic press (FIG. 14B) vs basket centrifuge (FIG. 14A)).

Dilute acid was subjected to rotary evaporation to remove water and to recover acid with a strength of 85 percent using bath temperature 75 degrees Celsius and vacuum in the range 220-20 mbar. Recovered acid will be reused for the biomass pretreatment from cycle 2 as described in the pre-treatment process. Comparison of recovered acid performance on biomass pretreatment by using two different types of solid/liquid separation methods, hydraulic press and basket centrifuge was evaluated.

It was observed that the basket centrifuge and hydraulic press provide similar results with regard to the amount of acid recovered for the recycle process. The results for Cycle 3 are shown in FIGS. 14A and 14B.

### Example 4

### Enzymatic Hydrolysis of Pretreated Biomass for Sugars Production

The system and method was used in Example 4 to evaluate enzymatic hydrolysis of pretreated biomass for sugars production. See also Example 2 (acid recovery).

Using the method and process described in Example 1. The recovered and concentrated acid from Cycle 3 was recycled into the process and used to conduct another treatment or pretreatment of the lignocellulosic biomass. The pretreated material was separated as in Example 1 followed by the recovery of ethanol, precipitation of lignin, and the concentration of the acid. The solid cake from the separation process was processed further to make sugar in the enzyme hydrolysis or sugar production process step. The acid was once again recovered as in Example 1 (e.g. evaporation of the solvent, precipitation of the lignin, and evaporation of the water making the Cycle 4 concentrated acid). The process step once again recovers and recycles about 85 percent to about 100 percent of the acid put into the treatment or pretreatment process step. The recovered and recycled acid seemed to have no significant impact on sugar production process yields. The data relating to sugars production generated while preparing the Cycle 4 acid is provided above in TABLES 3, 4, and 5.

### Overview of Exemplary Embodiments

Exemplary embodiments of the present inventions are shown and described schematically and representationally with reference to the FIGURES and TABLES.

As shown schematically in FIG. 1, a bio-refinery may comprise a biofuel and/or biochemical production facility according to an exemplary embodiment. The bio-refinery for biofuel and biochemical production may use a fermentation process. According to an exemplary embodiment, as shown schematically in FIG. 2, a bio-refinery may comprise a biofuel and/or biochemical production facility and a wood processing facility for producing biomass; the bio-refinery for biofuel and biochemical production may use a fermentation process (with biomass inputs) to process wood to biofuels and biochemicals. As shown schematically in FIG. 3, the production facility for producing biofuel and/or biochemical from biomass is configured for conversion of the biomass to prepared biomass in production of a biofuel and/or biochemical. See also FIGS. 4 and 5 (production of biofuel and/or biochemical from biomass according to exemplary embodiment).

FIGS. 6A through 6C are schematic block diagrams of systems for treating and processing the components from the (biofuel and/or biochemical) production facility indicating the process flow of the systems for the production of biofuel and/or biochemical from biomass is schematically according to an exemplary embodiment.

FIG. 7 is a schematic system block diagram of the system/subsystems for preparing and processing biomass (including pre-treatment and separation and treatment of liquid component and solids component) to produce biochemical and/or biofuel and to recover/recycle acid (for reuse in pre-treatment) according to an exemplary embodiment. As indicated schematically, the system comprises subsystems/apparatus to pre-treat the biomass (e.g. with concentrated acid such as phosphoric acid comprising recycled acid and fresh acid) and to separate the pre-treated biomass into a liquids component and a solids component; the system/subsystems and apparatus are configured to treat the liquid component (e.g. with a solvent such as ethanol and/or with water such as in a wash to remove lignin) and to recover and store concentrated acid (e.g. by treatment/separation such as filtration/evaporation/distillation) for reuse at pre-treatment; the system/subsystems and apparatus are configured to treat the solids component (e.g. to expose sugars for conversion/fermentation into a biofuel and to produce other biochemicals that can be extracted and/or produced) to produce biofuel (e.g. ethanol from evaporation/distillation) and biochemicals (e.g. compositions of various types selectively available from the process stream and/or residual). See also FIG. 1. According to an exemplary embodiment, lignin recovered or extracted from the process stream can be used as an energy source or otherwise. See also generally FIG. 1.

FIG. 8A is a schematic block diagram representing the system used for the preparation, pre-treatment and separation of biomass according to an exemplary embodiment; as indicated the process comprises pre-treatment with concentrated acid and separation of pre-treated biomass. See also FIG. 8B (example of an apparatus be used for pre-treating and separating the biomass according to an exemplary embodiment). As indicated schematically, a fermentation product derived from the biomass may be obtained by separating the pretreated biomass into (1) a liquid component comprising lignin, solvent and acid and (2) a solids component from which glucose can be made available for fermentation into the fermentation product (e.g. according to a fermentation process).

According to an exemplary embodiment as shown schematically in FIG. 9A, an apparatus may be used for the preparation, pre-treatment and separation of biomass, and recovering recoverable acid from the pre-treated biomass. See also FIG. 9B (an exemplary embodiment of an apparatus used for the pre-treatment and separation of biomass). As indicated schematically in FIG. 9B, the recovered acid (from components of pre-treatment/separation to obtain recoverable acid) is available to be used as contributed recovered acid (e.g. may be combined with fresh acid) as the concentrated acid for the pre-treatment/treatment operation for biomass. See also FIG. 7.

As shown schematically in FIG. 10 according to an exemplary embodiment, an apparatus may be used for treatment of a component (e.g. black liquor/liquid component) to recover ethanol applied in processing the pre-treatment/treated biomass may be a solvent (e.g. process ethanol).

As shown schematically in FIG. 11 according to an exemplary embodiment, an apparatus may be used for recovering from components (e.g. solids and/or liquid component) solids such as lignin and recoverable acid.

As shown schematically in FIG. 12 according to an exemplary embodiment, an apparatus may be used for re-concentrating recovered acid (e.g. the dilute acid stream) to be used as contributed acid.

FIGS. 13A through 13D indicate the operating conditions for the pre-treatment system according to an exemplary embodiment (e.g. operating conditions for the pre-treatment system for preparing the biomass into a prepared biomass).

FIG. 14A is a schematic graph representing according to an exemplary embodiment, the pre-treatment yield based on acid recovered from process cycle using basket centrifuge for the production of a fermentation product derived from the biomass to be obtained by separating the pretreated biomass into (1) a liquid component comprising lignin, solvent and acid and (2) a solids component from which glucose can be made available for fermentation into the fermentation product.

FIG. 14B is a schematic graph according to an exemplary embodiment, representing the pre-treatment yield based on acid recovered from process/cycle (e.g. using hydraulic press) for the production of a fermentation product derived from the biomass to be obtained by separating the pretreated biomass into (1) a liquid component comprising lignin, solvent and acid and (2) a solids component from which glucose can be made available for fermentation into the fermentation product.

FIG. 15 is a schematic graph according to an exemplary embodiment, representing the acid present (by weight) in the intermediate component (e.g. black liquor) in process/cycle based on separation, after pre-treatment (e.g. using basket centrifuge) (e.g. acid present (by weight) in the intermediate component (e.g. black liquor) in each cycle).

FIG. 16 is a schematic graph according to an exemplary embodiment, representing the acid present (by weight) in the water washes in process/cycle based on separation, after pre-treatment (e.g. using basket centrifuge) contributing recovered acid to be used as concentrated acid (e.g. acid present (by weight) in the water washes in each cycle).

FIG. 17 is a schematic graph according to an exemplary embodiment, showing the amount of acid recovered from intermediate component (e.g. black liquor) in process/cycle (e.g. using basket centrifuge) producing recovered acid from recoverable acid (e.g. showing the amount of acid recovered from intermediate component (e.g. black liquor) in process/cycle using basket centrifuge).

FIG. 18 is a schematic graph according to an exemplary embodiment, showing the amount of ethanol recovered from intermediate component (e.g. black liquor) in process/cycle (e.g. using basket centrifuge) showing the amount of ethanol recovered (e.g. recovered from intermediate component (e.g. black liquor) in each cycle using basket centrifuge).

FIG. 19 is a schematic graph according to an exemplary embodiment, showing the glucose yields from pretreated biomass in process/cycle (e.g. using basket centrifuge) facilitating the production of a fermentation product derived from the biomass to be obtained by separating the pretreated biomass into (1) a liquid component comprising lignin, solvent and acid and (2) a solids component from which glucose can be made available for fermentation into the fermentation product.

FIG. 20 is a schematic graph showing overall sugar yields from pretreated biomass based on process/cycle (e.g. using basket centrifuge) facilitating the production of a fermentation product derived from the biomass to be obtained by separating the pretreated biomass into (1) a liquid component comprising lignin, solvent and acid and (2) a solids component from which glucose can be made available for fermentation into the fermentation product according to an exemplary embodiment.

According to an exemplary embodiment, a method to pretreat biomass to be used in a biorefinery to produce a biofuel or biochemical using a fermentation process may comprise the steps of preparing the biomass into prepared biomass; pre-treating the prepared biomass into a pretreated biomass by application of a concentrated acid; facilitating the production of a fermentation product derived from the biomass to be obtained by separating the pretreated biomass into a liquid component comprising lignin, solvent and acid and a solids component from which glucose can be made available for fermentation into the fermentation product; recovering recoverable acid from the pre-treated biomass; producing recovered acid from recoverable acid; and contributing recovered acid to be used as concentrated acid. According to an exemplary embodiment, the biomass may comprise lignocellulosic material; the lignocellulosic material may be derived from a wood material.

According to an exemplary embodiment, the method may comprise the step of treatment of the pre-treated biomass; the method may comprise the step of separation of the pre-treated biomass; the method may comprise the step of treatment of the liquid component. See e.g. FIGS. 1, 2-4 and 5-7. Treatment may comprise adding solvent to the pretreated biomass; treatment may comprise adding water to the pretreated biomass.

According to an exemplary embodiment, the method may comprise the step of separation of the liquid component and pretreated biomass; separation may comprise filtration; separation may comprise filtration to remove lignin and to separate the cellulosic solids from the liquid phase. See e.g. FIGS. 1, 5-7, 8A-8B, 9A-9B, 10-12. Separation may comprise filtration, evaporation and/or distillation. Separation may comprise evaporation to remove solvent; separation may comprise a separation of the liquid component. Separation may comprise distillation to recover recoverable acid. According to an exemplary embodiment, the method may comprise the step of treatment of the solid component; treatment may comprise at least one wash. According to an exemplary embodiment, the method may comprise the step of separation of the solid component.

The treatment may comprise dilution of the liquid component; the treatment may comprise dissolving lignin and acid with solvent. See e.g. FIGS. 7 and 9A-9B. According to an exemplary embodiment, water may be added at a ratio from about 1:1 part to 5:1 part water to one part liquid component after evaporation; water may be added at a ratio from about 1:1 part to 5:1 part water to one part liquid component after evaporation; water may be added at a ratio of about 1:1 to about 3:1 water to liquid component after evaporation. According to an exemplary embodiment, water may be added at a ratio of about 1:1 to about 3:1 water to liquid component after evaporation; water may be added at a preferred ratio of about 2:1 to about 1:1 water to liquid component after evaporation; water may be added at a preferred ratio of about 2:1 to about 1:1 water to liquid component after evaporation.

According to an exemplary embodiment, the separation may comprise separation of recoverable acid; the separation may comprise separation of recoverable acid; recoverable acid may be recovered from acid in the liquid component. See e.g. FIGS. 1, 7, 11-12. Separation may comprise evaporation, distillation and/or filtration. The liquid component may be evaporated to recover solvent.

According to an exemplary embodiment, treatment may comprise application of solvent; treatment may comprise application of water; application may comprise a wash. See e.g. FIGS. 1, 7 and 11. The solvent may comprise ethanol. Water may be added to the liquid component after the solvent is removed. According to an exemplary embodiment, recoverable acid may be recovered from the solids component. According to an exemplary embodiment, the concentrated acid may be provided with a concentration in a range of 75 to 100 percent; the concentrated acid may comprise at least between 90 to 100 percent recovered acid; at least 98 percent recovered acid. The concentrated acid may comprise at least between 0 to 10 percent fresh acid; at least 2 percent fresh acid; fresh acid comprises acid not previously used to treat biomass. According to an exemplary embodiment, recovered acid may comprise acid recovered from pre-treated biomass.

According to an exemplary embodiment, pre-treating the biomass may be performed at operating conditions comprising a temperature in a range of about 20 to about 90 degrees Celsius for a period of time in a range of about 15 minutes to 4 hours.

According to an exemplary embodiment, the wood material may comprise at least one of oak wood, yellow poplar, maple, hickory and other indigenous hard woods to North America. According to an exemplary embodiment, the lignocellulosic material may comprise 30 to 55 percent cellulose by weight and hemicellulose at about 15 to 40 percent by weight; the lignocellulose material may comprise glucan; the glucan may comprise cellulose. The lignocellulosic material may comprise xylan; the hemicellulose may comprise xylan.

According to an exemplary embodiment, the liquid component may comprise mother liquor; the concentrated acid may comprise phosphoric acid. The liquid component may comprise about 10 to 20 percent phosphoric acid; the liquid component may comprise about 40 to 60 percent solvent. The liquid component may comprise about 0 to 5 percent xylose; the liquid component may comprise about 0 to 2 percent glucose. The liquid component may comprise recoverable lignin. The mother liquor may comprise about 1 to 75 percent lignin by weight of the solids; the mother liquor may comprise about 50 percent lignin by weight of the solids. The liquid component may comprise lignin in a percentage of at least 50 percent of the lignin in the lignocellulosic material. According to an exemplary embodiment, treatment may comprise addition of water to cause lignin to precipitate; treatment may comprise use of a solvent; the solvent may comprise ethanol; evaporation may be used to separate the solvent from the acid-lignin mixture.

According to an exemplary embodiment, lignin may be recovered using a liquid-solid separation unit operation; the liquid-solid separation unit operation may use a plate and frame filter press; the liquid-solid separation unit operation may use a basket centrifuge.

According to an exemplary embodiment, the liquid after lignin removal may be subjected to evaporation to remove the water and re-concentrate the phosphoric acid. The phosphoric acid may be re-concentrated to about 75 percent on a weight basis; the phosphoric acid may be re-concentrated to about 80 percent on a weight basis; the phosphoric acid may be re-concentrated to about 85 percent on a weight basis; the phosphoric acid may be re-concentrated to about 90 percent on a weight basis.

According to an exemplary embodiment, the process streams may comprise a liquid component and a solids component (see FIG. 7); solids component may be treated/separated with a wash or series of washes that recover effectively the majority of acid (e.g. first water wash recovers all but 5-10 percent of the acid; second wash recovers all but 2-3 percent of the acid; third wash recovers all but about 1 percent of the acid; fourth/subsequent washes (if feasible) may recover virtually all remaining acid as recovered/recoverable acid to be used for concentrated acid in the pre-treatment process); if ethanol is used as a solvent according to an exemplary embodiment the ethanol will be removed from solids which may be processed with enzymes (or contain other recoverable compositions or material/matter than is sensitive to ethanol or solvent). Water may be used in process (e.g. as a wash, to precipitate lignin, etc.).

The system and method according to an exemplary embodiment may employ subsystems for treatments (e.g. washes, etc.) and separations (e.g. filtration, evaporation, distillation). See FIGS. 1 and 7 and 9A-9B, 10-12.

According to an exemplary embodiment, the wood material may comprise hard wood sawdust consisting of at least oak wood; the wood material may comprise at least one of oak wood, yellow poplar, maple, hickory and other indigenous hard woods to North America. According to an exemplary embodiment, the lignocellulosic material may comprise 30 to 55 percent cellulose by weight and hemicellulose at about 15 to 40 percent by weight; the lignocellulosic material may comprise oak hard wood saw dust and may comprise cellulose at about 35 to 45 percent cellulose by weight and hemicellulose at about 17 to 24 percent by weight. According to an exemplary embodiment, the lignocellulose material comprises or consists essentially of oak wood, maple wood, and yellow poplar wood or an agricultural residue; lignocellulosic material may be saw dust residue from a wood processing facility. According to an exemplary embodiment, the lignocellulose material may be an agricultural residue consisting essentially of corn stover, wheat straw, oat straw, rice straw, rape seed stray, grass, sugar cane bagasse, sugar beet bagasse, empty fruit bunches from palm oil extraction and/or other agricultural residue; the lignocellulosic material may comprise oak wood.

A system and method to pre-treat and treat biomass to make bioproducts such as biofuels and biochemical may be configured to treat bioproduct streams such as comprising a liquid component separated from biomass to yield sugars available to produce a fermentation product or bio-product and a solids component separated from biomass. The system and method comprises treatment and separation of process streams (e.g. liquids, solids, etc.) to produce bioproducts such as fuel and biochemical as well as to recover/reuse and utilize material for processing. Lignin is separated from the bioproduct stream (e.g. a solids component) forming a recovered bioproducts; efficient removal of lignin facilitates productive operation of the system. The system disclosed is configured to recover and recycle acid (e.g. phosphoric acid) used as a concentrated acid in the process to pre-treat biomass (for bioprocessing). The acid used in the process is recycled and recovered in order to establish economically attractive biorefinery to make bio-products from biomass (e.g. to reduce the cost of operation such as from supply of fresh acid). Concentrated acid used to pre-treat biomass may comprise about 90 to over 98 percent recovered acid (and 10 to less than 2 percent fresh acid).

The embodiments as disclosed and described in the application (including the FIGURES, TABLES and Examples) are intended to be illustrative and explanatory of the present inventions. Modifications and variations of the disclosed embodiments, for example, of the apparatus and processes employed (or to be employed) as well as of the compositions and treatments used (or to be used), are possible; all such modifications and variations are intended to be within the scope of the present inventions.

It is important to note that the construction and arrangement of the elements of the inventive concepts and inventions as described in this application and as shown in the figures above is illustrative only. Although some embodiments of the present inventions have been described in detail in this disclosure, those skilled in the art who review this disclosure will readily appreciate that many modifications are possible without materially departing from the novel teachings and advantages of the subject matter recited. Accordingly, all such modifications are intended to be included within the scope of the present inventions. Other substitutions, modifications, changes and omissions may be made in the design, operating conditions and arrangement of the preferred and other exemplary embodiments without departing from the spirit of the present inventions.

It is important to note that the system and method of the present inventions can comprise conventional technology (e.g. as implemented in present configuration) or any other applicable technology (present or future) that has the capability to perform the functions and processes/operations indicated in the FIGURES. All such technology is considered to be within the scope of the present inventions and application.

1. A method to pretreat biomass to be used in a biorefinery to produce a biofuel or biochemical using a fermentation process comprising the steps of:
(a) preparing the biomass into prepared biomass;
(b) pre-treating the prepared biomass into a pretreated biomass by application of a concentrated acid;
(c) facilitating the production of a fermentation product derived from the biomass to be obtained by separating the pretreated biomass into (1) a liquid component comprising lignin, solvent and acid and (2) a solids component from which glucose can be made available for fermentation into the fermentation product;
(d) recovering recoverable acid from the pre-treated biomass;
(e) producing recovered acid from recoverable acid; and
(f) contributing recovered acid to be used as concentrated acid;
wherein the biomass comprises lignocellulosic material; and
wherein the lignocellulosic material is derived from a wood material.

2. The method of claim 1, further comprising the step of treatment of at least one of (a) the liquid component or (b) the pre-treated biomass.

3. The method of claim 2, wherein treatment comprises adding solvent or water to the pretreated biomass.

4. The method of claim 1, further comprising the step of separation of at least one of (a) the liquid component or (b) pretreated biomass, wherein, preferably, separation comprises at least one of (a) evaporation; (b) distillation; (c) filtration; (d) distillation to recover recoverable acid; (e) separation of the solids component; (f) separation of recoverable acid; (g) filtration to remove lignin and other solids from the liquid component; (h) evaporation to remove water or solvent; or (i) separation of recoverable acid; and wherein recoverable acid is recovered from at least one of the liquid component or the solid component.

5. The method of claim 1, further comprising the step of treatment of the solid component; and wherein treatment comprises at least one of (a) at least one wash; (b) dilution of the liquid component; or (c) dissolving lignin and acid with solvent.

6. The method of claim 1, wherein the liquid component is evaporated to recover solvent.

7. The method of claim 1, wherein treatment comprises of at least one of (a) application of solvent; (b) application of water; (c) addition of water to cause lignin to precipitate; wherein solvent comprises ethanol; wherein application comprises a wash.

8. The method of any of claim 3, wherein water is added (a) at a ratio from about 1:1 part to 5:1 part water to one part liquid component after evaporation; (b) at a ratio of about 1:1 to about 3:1 water to liquid component after evaporation.

9. The method of claim 1, wherein the concentrated acid comprises phosphoric acid, and/or wherein the liquid component comprises about 10 to 20 percent phosphoric acid, and/or wherein the liquid component comprises about 40 to 60 percent solvent, and/or wherein the liquid component comprises about 0 to 5 percent xylose and about 0 to 2 percent glucose, and/or wherein the liquid component comprises about 1 to 75 percent lignin by weight of the solids.

10. The method of claim 1, wherein the liquid component after lignin removal is subjected to evaporation to remove the water and re-concentrate the phosphoric acid for use as concentrated acid.

11. The method of claim 3, wherein water is added to the liquid component after the solvent is removed.

12. The method of claim 1, wherein the concentrated acid comprises recovered acid and fresh acid; wherein fresh acid comprises acid not previously used to treat biomass; and wherein recovered acid comprises acid recovered from pre-treated biomass.

13. The method of claim 1, wherein the concentrated acid is provided with a concentration in a range of 75 to 100 percent; and wherein the concentrated acid comprises at least between 90 to about 100 percent recovered acid and between 0 to 10 percent fresh acid.

14. The method of claim 1, wherein pre-treating the biomass is performed at operating conditions comprising a temperature in a range of about 20 to about 90 degrees Celsius for a period of time in a range of about 15 minutes to 4 hours.

15. The method of claim 1, wherein the lignocellulosic material comprises 30 to 55 percent cellulose by weight and hemicellulose at about 15 to 40 percent by weight; wherein the lignocellulose material comprises glucan and xylan; and wherein the glucan comprises cellulose; wherein the hemicellulose comprises xylan, and/or wherein the lignocellulosic material comprises at least one of (a) a wood material; or (b) saw dust residue (an agricultural residue); and wherein the wood material comprises at least one of saw dust, saw dust residue; hard wood sawdust consisting of at least oak wood residue from a wood processing facility, oak wood, maple wood, hickory wood, poplar wood, other indigenous hard woods to North America; and wherein agricultural residue comprises at least one of corn stover, wheat straw, oat straw, rice straw, sugar cane bagasse, sugar beet bagasse, empty fruit bunches from palm oil extraction, other agricultural residue, and/or wherein the lignocellulosic material comprises wood residue and comprises cellulose at about 35 to 45 percent cellulose by weight and hemicellulose at about 17 to 24 percent by weight.

16. The method of claim 1, wherein separation is performed in a liquid-solid separator unit comprising at least one of (a) a plate and frame filter press or (b) a basket centrifuge.
